SEQUENCE LISTING (12 sequences)

SEQ ID NO: 1 - DNA, 27 bp, Artificial sequence
  Synthetic oligonucleotide
gacgccgctgtcgccccagaggagcgc

SEQ ID NO: 2 - DNA, 27 bp, Artificial sequence
  Synthetic oligonucleotide
gcgctcctctggggcgacagcggcgtc

SEQ ID NO: 3 - DNA, 33 bp, Artificial sequence
  Synthetic oligonucleotide
gttgacgccgctgtcgaaccagaggagcgccac

SEQ ID NO: 4 - DNA, 33 bp, Artificial sequence
  Synthetic oligonucleotide
gtggcgctcctctggttcgacagcggcgtcaac

SEQ ID NO: 5 - DNA, 18 bp, Artificial sequence
  Synthetic oligonucleotide
atctccagccgtggcatt

SEQ ID NO: 6 - DNA, 18 bp, Artificial sequence
  Synthetic oligonucleotide
ttcgtagccgttctgctg

SEQ ID NO: 7 - protein, 47 aa, homo sapiens
KKKQYTSIHHGVVEVDAAVTPEERHLSKMQQNGYENPTYKFFEQMQN

SEQ ID NO: 8 - protein, 47 aa, homo sapiens
RKRQYGTISHGIVEVDPMLTPEERHLNKMQNHGYENPTYKYLEQMQI

SEQ ID NO: 9 - protein, 47 aa, homo sapiens
RRKKPYGAISHGVVEVDPMLTLEEQQLRELQRHGYENPTYRFLEERP

SEQ ID NO: 10 - protein, 6 aa, homo sapiens
YENPTY

SEQ ID NO: 11 - protein, 4 aa, Artificial sequence
  Synthetic polypeptide
NPXY

SEQ ID NO: 12 - protein, 7 aa, Homo sapiens
GYENPTY

(19) United States
(12) Patent Application Publication
(10) Pub. No.: US 2010/0311726 A1
(43) Pub. Date: Dec. 9, 2010

(54) REDUCTION OF BETA-AMYLOID LEVELS BY TREATMENT WITH THE SMALL MOLECULE DIFFERENTIATION-INDUCING FACTOR

(76) Inventors: Michael Myre, Salem, MA (US); Wilma Wasco, Arlington, MA (US); Rudolph E. Tanzi, Hull, MA (US)

Correspondence Address: WOLF GREENFIELD & SACKS, P.C., 600 ATLANTIC AVENUE, BOSTON, MA, 02210-2206, US

(21) Appl. No.: 12/669,101

(22) PCT Filed: Jul. 16, 2008
(86) PCT No.: PCT/US08/08688

§ 371 Date: Jun. 21, 2010

Related U.S. Application Data
(60) Provisional application No. 60/959,646, filed on Jul. 16, 2007.

(51) Int. Cl. A61K 31/12; A61K 31/445; A61K 31/27; A61K 31/55; A61K 31/4355; A61K 31/13; A61K 31/498; A61P 25/00; A61P 25/28

(52) U.S. Cl. 514/215; 514/678; 514/319; 514/479; 514/297; 514/662; 514/249; 514/291

(57) ABSTRACT

The present invention relates to novel uses for a family of small molecules, Differentiation-Inducing Factors (DIFs). It has been discovered that DIFs surprisingly can alter the metabolic processing of amyloid precursor protein (APP) and in turn reduce the level of secreted Aβ. The metabolic processing of other γ-secretase substrates normally present in cells (Notch and APLP1) is not affected when treated with DIF. The invention provides methods for reducing Aβ production in mammalian cells that express APP by administering DIF-I, DIF-2, DIF-3, a functionally equivalent analog and or any combination thereof. The invention also provides methods for treating and/or preventing Alzheimer's disease by preferentially reducing Aβ production.

### RELATED APPLICATION

This application claims the benefit under 35 U.S.C. §119(e) of U.S. provisional application 60/959,646, filed Jul. 16, 2007, the entire disclosure of which is incorporated herein by reference.

### GOVERNMENT INTEREST

This work was funded in part by the National Institutes of Health under grant number AG 15379. The government has certain rights in this invention.

### FIELD OF THE INVENTION

The present invention relates to the treatment and prophylaxis prevention of Alzheimer's disease. More specifically, the invention relates to methods for reducing production of Aβ by inhibiting γ-secretase using the small molecule, Differentiation-Inducing Factor-1 (DIF-1).

### BACKGROUND OF THE INVENTION

Alzheimer's Disease (AD) is a degenerative brain disorder characterized clinically by progressive loss of memory, cognition, reasoning, judgment and emotional stability that gradually leads to profound mental deterioration and ultimately death. AD is a very common cause of progressive mental failure (dementia) in aged humans and is believed to represent the fourth most common medical cause of death in the United States. AD has been observed in races and ethnic groups worldwide and presents a major present and future public health problem. The disease is currently estimated to affect about two to three million individuals in the United States alone. AD is at present incurable. No treatment that effectively prevents AD or reverses its symptoms and course is currently known.

The brains of individuals with AD exhibit characteristic lesions termed senile (or amyloid) plaques, amyloid angiopathy (amyloid deposits in blood vessels) and neurofibrillary tangles. Large numbers of these lesions, particularly amyloid plaques and neurofibrillary tangles, are generally found in several areas of the human brain important for memory and cognitive function in patients with AD. Smaller numbers of these lesions in a more restrictive anatomical distribution are also found in the brains of most aged humans who do not have clinical AD. Amyloid plaques and amyloid angiopathy also characterize the brains of individuals with Trisomy 21 (Down's Syndrome) and Hereditary Cerebral Hemorrhage with Amyloidosis of the Dutch Type (HCHWA-D). At present, a definitive diagnosis of AD usually requires observing the aforementioned lesions in the brain tissue of patients who have died with the disease or, rarely, in small biopsied samples of brain tissue taken during an invasive neurosurgical procedure.

The principal protein implicated in the development of Alzheimer's disease is the Amyloid Precursor Protein (APP). APP is as an integral transmembrane protein that is widely expressed on the surface of cells particularly in neurons. APP has a 40-42 amino acid domain, termed Aβ, which is the main component of the AD-associated amyloid plaques. Genetic, neuropathological, and biochemical findings indicate that excessive production and/or accumulation of Aβ peptide play a fundamental role in the pathogenesis of AD. This has led to the proposed amyloid cascade hypothesis, which posits that altered amyloid processing/aggregation constitute the key pathogenic factor in AD, and that the rest of the disease is due to amyloid-induced toxicity.

Regulation of APP processing is complex. Cleavage of APP by either α- or β-secretases produces soluble N-terminal fragments sAPPα and sAPPβ, and smaller C83 and C99 membrane-bound C-terminal fragments (CTFs). The CTFs can be further cleaved by γ-secretase leading to the secretion of the non-pathogenic p3 peptide and Aβ. The non-amyloidogenic cleavage of APP by α-secretase is a major pathway of ‘normal’ APP metabolism, and agents that shift cellular metabolism towards a-cleavage of APP lead to a significant decrease in Aβ.

BACE1 has been shown to act as the major β-secretase. β-secretase is a type I transmembrane, glycosylated aspartyl protease found in post-Golgi membranes and at the cell surface and can hydrolyze APP in the extracellular domain, either between Met671 and Asp672, (or between residues 682 and 683). This cleavage by β-secretase generates a 99-residue membrane-associated C-terminus fragment (APP-C99). APP-C99 is further cleaved to release 4-kDa Aβ and β-amyloid precursor protein intracellular domain (AICD). This cleavage is achieved by an unusual form of proteolysis in which the protein is cleaved within the transmembrane domain (at residue +40 or +42) by γ-secretase.

APP is more routinely cleaved by α-secretase, at the site close to the transmembrane domain and in the middle of the Aβ region of APP, to release a large ectodomain (α-APPs), leaving a carboxy-terminus fragment of 83 amino acids (APP-C83) in the membrane. While proteolysis of APP-C99 by γ-secretase produces Aβ, proteolysis of APP-C83 by γ-secretase produces p3, a peptide resembling an amino-terminally truncated form of Aβ. Presenilin (PS) and γ-secretase co-fractionate as a detergent-sensitive, high molecular weight complex that includes at least three other proteins, nicastrin/APH2, APH-1, and PEN-2, all of which are necessary for γ-secretase activity.

Clearly, one of the benefits of understanding the mechanisms involved in APP processing will be the identification of new areas to target for drug intervention and ultimately the prevention of AD. Presenilin (PS) and γ-secretase co-fractionate as a detergent-sensitive, high molecular weight complex that includes at least three other proteins, nicastrin/APH2, APH-1 A and B, and PEN-2, all of which are necessary for γ-secretase activity. APP-CTFs comprise a mixture of substrates that can be processed by multiple proteolytic pathways, as for instance the γ-secretase or the proteasome pathway.

Despite the progress which has been made in understanding the underlying mechanisms of the pathogenesis of AD and other Aβ peptide related diseases, there remains a need to develop methods and compositions for treatment of the disease(s). Ideally, the treatment methods would advantageously be based on drugs which are capable of inhibiting Aβ peptide release and/or its synthesis in vivo.

Current treatments for Alzheimer's disease offer at best modest efficacy; thus, there is a need for a therapy able to alter the disease's pathophysiology (Hardy and Selkoe, 2002). The pathogenesis of AD is linked to abnormal processing of amyloid precursor protein, or APP, phosphoprotein highly expressed in the brain. It is thought that abnormality in APP processing leads to, at least in part, accumulation of a toxic end product, Aβ (also known as β-amyloid), which ultimately results in the formation of neurofibrillary plaques in affected brains. Therefore, it is of much interest to understand the mechanisms underlying the process of APP regulation and develop an effective and safe therapeutics that pose minimal side effects.

### SUMMARY OF THE INVENTION

Alzheimer's disease (AD) is one of the greatest public health problems in the U.S., and its impact will only increase in coming decades. AD, an insidious and progressive neurodegenerative disorder accounting for the vast majority of dementia, is characterized by global cognitive decline and the robust accumulation of amyloid deposits and neurofbrillary tangles in the brain. Effective treatments for AD are lacking while current AD treatments, focusing on downstream events in AD neuropathogenesis, afford only modest, temporary, and palliative benefit. This invention describes a novel use for the small molecule, DIF-1 and analogs thereof, that can be used for therapies aimed at treating and preventing AD through the lowering of Aβ levels via interfering with the posttranslational phosphorylation of amyloid precursor protein (APP).

The present invention is based on novel uses for a family of small molecules, Differentiation-Inducing Factors (DIFs). It has been surprisingly discovered that DIFs can alter the metabolic processing of APP and in turn reduce the level of secreted Aβ. In addition, the inhibitory effect of DIFs on APP processing is reversible. Importantly, the metabolic processing of other γ-secretase substrates normally present in cells (e.g., Notch and APLP1) is not affected when treated with DIF.

The invention in one aspect provides methods for treating or preventing a disease in which Aβ is a causative factor or symptom. The methods include administering to a subject in need of such treatment a composition comprising DIF-1, an analog thereof, a salt thereof, a solvate thereof or any combination thereof, in an amount effective to reduce Aβ production. In some embodiments, the disease in which A13 is a causative factor or symptom is Alzheimer's disease (AD), Down's syndrome, multi-infarct dementia, dementia puglistica, cerebrovascular amyloidosis (Cerebral Amyloid Angiopathy), Hereditary Amyloidosis with Cerebral Hemorrhage of the Dutch Type (HCHWA-D), Familial British Dementia, vascular dementia, and inclusion body myositis, or homozygocity for the apolipoprotein E4 allele. In a preferred embodiment the disease is Alzheimer's disease.

In any of the above embodiments, the subject is preferably a human.

In some of the above embodiments, the subject is otherwise free of symptoms calling for treatment with the agent. In some of the above embodiments, the subject does not have a cancer. In some of the above embodiments, the subject is apparently healthy. In yet other of the above embodiments, the subject exhibits one or more symptoms of a disease in which Aβ is a causative factor or symptom. For example, the subject may exhibit one or more symptoms of Alzheimer's disease (AD), Down's syndrome, multi-infarct dementia, dementia puglistica, cerebrovascular amyloidosis (Cerebral Amyloid Angiopathy), Hereditary Amyloidosis with Cerebral Hemorrhage of the Dutch Type (HCHWA-D), Familial British Dementia, vascular dementia, and inclusion body myositis, or homozygocity for the apolipoprotein E4 allele.

In any of the embodiments provided above, Aβ production is reduced by at least 10%, preferably at least 20%, or more preferably 50%.

Any of the methods provided above may further comprise administering an Alzheimer's disease treatment. In some embodiments, the Alzheimer's disease treatment is a cholinesterase inhibitor, such as donepezil (Aricept®), rivastigmine (Exelon®), galantamine (Remix®), or tacrine (Cognex®); a NMDA receptor antagonist, such as memantine (Namenda®); an AMPA receptor agonist, such as CX516 (Ampalex®); a choline uptake enhancer, such as MKC-231; a HMG CoA reductase inhibitor, such as a statin; or immune therapy.

In certain of the foregoing embodiments, the DIF-1, analog thereof, salt thereof, solvate thereof, Alzheimer's disease therapeutic, or combination thereof is administered orally, intravenously, intramuscularly, intranasally, intraperitoneally, subcutaneously, or intrathecally.

According to a second aspect of the invention, methods for reducing Aβ production in a mammalian cell are provided. The methods for reducing Aβ production in a mammalian cell include contacting a mammalian cell expressing APP with DIF-1, an analog thereof, a salt thereof, a solvate thereof, or any combination thereof, in an amount effective to reduce Aβ production.

In some embodiments, the amount of the DIF-1, the analog thereof, the salt thereof, the solvate thereof, or any combination thereof, is an amount effective to reduce γ-secretase-dependent proteolysis of APP preferentially as compared to γ-secretase-dependent proteolysis of other cellular substrates of γ-secretase. In some embodiments, the other cellular substrates of γ-secretase are APLP1 and/or Notch.

In some preferred embodiments, the mammalian cell is contacted with DIF-1, the salt thereof or the solvate thereof. In other preferred embodiments, the mammalian cell is contacted with the analog of DIF-1, the salt thereof or the solvate thereof.

In further embodiments, the invention provides methods which further include contacting the mammalian cell with an Alzheimer's disease treatment. In some embodiments, the Alzheimer's disease treatment includes a cholinesterase inhibitor, such as donepezil (Aricept®), rivastigmine (Exelon®), galantamine (Remix®), or tacrine (Cognex®); a NMDA receptor antagonist, such as memantine (Namenda®); an AMPA receptor agonist, such as CX516 (Ampalex®); a choline uptake enhancer, such as MKC-231; a HMG CoA reductase inhibitor, such as a statin; or immune therapy.

In some embodiments, Aβ production is reduced by at least 10%. Preferably, Aβ production is reduced by at least 20%. More preferably, Aβ production is reduced by at least 50%.

In a third aspect of the invention, pharmaceutical formulations are provided. The pharmaceutical formulations include an amount of (1) DIF-1, an analog thereof, a salt thereof, a solvate thereof or any combination thereof, and an amount of (2) an Alzheimer's disease treatment.

In some embodiments, the pharmaceutical formulations further include a pharmaceutically acceptable carrier.

In some embodiments, the pharmaceutical formulation comprises DIF-1, a salt thereof or a solvate thereof. In other embodiments, the pharmaceutical formulation comprises an analog of DIF-1, a salt thereof or a solvate thereof.

In some embodiments, the Alzheimer's disease treatment is a cholinesterase inhibitor, such as donepezil (Aricept®), rivastigmine (Exelon®), galantamine (Remix®), or tacrine (Cognex®); a NMDA receptor antagonist, such as memantine (Namenda®); an AMPA receptor agonist, such as CX516 (Ampalex®); a choline uptake enhancer, such as MKC-231; a HMG CoA reductase inhibitor, such as a statin; or immune therapy.

In some of the preceding embodiments, the amount of (1) and the amount of (2) are in a single formulation or unit dosage form.

In some embodiments, the formulation or unit dosage form is an oral formulation or unit dosage form, such as a liquid, a syrup, a tablet, a capsule, a powder, a sprinkle, a chewtab, or a dissolvable disc.

According to a fourth aspect, the invention provides methods for reducing APP phosphorylation. The methods include contacting APP with DIF-1, an analog thereof, a salt thereof, a solvate thereof or any combination thereof, in an amount effective to reduce APP phosphorylation at the amino acid residue 668 (Thr668). In some embodiments, the APP is contacted with DIF-1, the salt thereof, or the solvate thereof. In further embodiments, the APP is contacted with an analog of DIF-1, the salt thereof, or the solvate thereof.

In a fifth aspect, the invention provides methods for preferentially inhibiting APP processing by γ-secretase. The methods include contacting γ-secretase with DIF-1, an analog thereof, or any combination thereof, in an amount effective to preferentially inhibit APP processing by γ-secretase as compared to processing by γ-secretase of non-APP substrates of γ-secretase, wherein the amount effective to preferentially inhibit APP processing by γ-secretase does not significantly inhibit one or more non-APP substrates of γ-secretase. According to some embodiments, the one or more non-APP substrates of γ-secretase comprises APLP1 and/or Notch.

In another aspect, the invention provides for use of the foregoing agents, compounds and molecules in the preparation of medicaments, particularly medicaments for the treatment of Alzheimer's disease and other conditions in which reduced production of Aβ is therapeutically or prophylactically beneficial.

These and other aspects of the invention will be described in greater detail below.

### BRIEF DESCRIPTION OF THE DRAWINGS

FIG. 1 provides three graphs showing antiproliferative effect of DIF-1 on CHO cells: (A) The time course of the increase in cell numbers; and (B) Flow cytometry.

FIG. 2 provides three graphs showing antiproliferative effect of DIF-1 on CHO/APP cells; (A) The time course of the increase in cell numbers; and (B) Flow cytometry.

FIG. 3 provides a set of Western blot images demonstrating reduced expression of cyclin D1 in CHO/APP⁷⁵¹ cells treated with 30 μM DIF-1 or DIF-1 plus DAPT (250 nM). Top: cyclin D1 levels; Bottom: GAPDH loading control. Molecular weight of cyclin D1 is shown on the left. This blot is representative of at least 3 separate experiments using CHO-7W cells.

FIG. 4 provides a Western blot image showing the effect of 30 μM DIF-1 on APP expression in CHO-7W cell RIPA lysates. Molecular weights are shown on the left. Vehicle is ethanol. Results are representative of at least five independent experiments.

FIG. 5 presents a set of Western blot images showing that the DIF-1 effect on APP expression in CHO-7W cells is not blocked by the addition of the potent γ-secretase inhibitor DAPT. Top: full length APP; Middle: APP CTFs; Bottom: GAPDH loading control. Molecular weight markers are shown on the left. Results are representative of at least five independent experiments.

FIG. 6 provides a set of Western blot images showing that the DIF-1 effect on APP expression in CHO-CAB cells is also not blocked by the addition of DAPT. Bottom: GAPDH loading control. Molecular weight markers are shown on the left. Results are representative of at least five independent experiments.

FIG. 7 provides set of Western blot images showing the effect of DIF-1 on APLP 1 metabolism in the presence or absence of DAPT. Each treatment is shown above the blot. Top right: full length APLP1 levels; Middle right: APLP1 CTF levels; Bottom right: GAPDH loading control levels. Molecular weight markers are shown on the left. Results are representative of three independent experiments.

FIG. 8 depicts Clustal alignment for the comparison of the last 47 amino acids of human APP [SEQ ID NO:7], APLP2 [SEQ ID NO:8] and APLP1 [SEQ ID NO:9]. The highly conserved YENPTY motif [SEQ ID NO:10] is highlighted in medium gray. A predicted proline-directed class IV WW phosphorylation-dependent interaction motif is highlighted in light gray (S/TP). APLP1 contains a leucine (L) residue following the threonine (T) and is highlighted in dark gray. Identical residues between all three C-terminal domains are denoted with an asterisk (*), where as homologous residues are denoted with a colon (:) below the alignment.

FIG. 9 provides three sets of Western blot images, showing: (A) the dose-dependent effect of DIF-1 on APP processing; (B) Analysis of conditioned media for secreted Aβ and total secreted soluble APP by western blot (Soluble αAPP levels and Aβ levels are shown with arrows on the left. Treatments are labeled on the top of the blot); and (C) Analysis of conditioned media for total secreted soluble APP from CHO-CAB cells treated with 30 μM DIF-1. Molecular weight markers are shown on the left. Treatments are labeled on top of the blot. The blots are representative of three separate experiments.

FIG. 10 provides two graphs showing the DIF-1 effects on levels of secreted Aβ as measured by sandwich ELISA: (A) Comparison of the amount of Aβ40 and Aβ42 as measured by sandwich ELISA in CHO-7W conditioned media; (B) Dose-dependent effect of DIF-1 on the levels of secreted Aβ40 and Aβ42.

FIG. 11 provides four Western blot images, showing that DIF-1 (30 μM) reduces the level of APP phosphorylation on residue T668: (A) Cells treated with DAPT, and DAPT+DIF probed with anti-APP antibody (C66). Molecular weight markers are shown on the left. All treatments are labeled above the blot; (B) CHO-7W cells treated with DAPT, and DAPT+DIF, and then probed with polyclonal anti-P668 APP antibody (1:1000); (C) CHO-CAB cells; and (D) H4 cells expressing APP^{sw}.

FIG. 12 provides a set of Western blot images showing the effect of LiCl on DIF-1 mediated APP metabolism. Lithium treatments alone caused increases in both αCTF and βCTFs (arrowhead) whereas pretreatment with lithium prior to DIF-1 caused only a minor increase in the level of αCTF (arrowhead). Lithium treatment did not significantly prevent the effect of DIF-1 on APP maturation (arrowhead). U=untreated cells; EtOH=ethanol vehicle control. GAPDH loading controls are shown below. Molecular Weight markers (kDa) are shown on the left.

FIG. 13 provides a set of Western blot images, showing that DIF-1 reverses proteasome-mediated accumulation of APP CTF levels: (A) Effects of GSK inhibitors on DIF-1 processing of APP; (B) The effect of proteasome inhibitors on DIF-1 processing of APP; (C) The effect of a second proteasome inhibitor, ALLN, on APP processing; and, (D) The effect of DIF-1 on the cellular level of APP ubiquitin-conjugates in CHO-CAB cells.

FIG. 14 provides a panel of confocal microscopy images, showing that DIF-1 alters the subcellular distribution of APP. (A) Detection of APP⁷⁵¹ in stably transfected CHO-CAB cells; and, (B) Treatment with DIF-1 increased the detection of APP around the nuclear periphery where the staining appears as large, punctuate spots. The upper panel is a representation of APP staining upon treatment with DIF-1.

FIG. 15 provides a panel of confocal microscopy images, showing that DIF-1 increases the co-localization of cellular APP and ubiquitin in CHO-CAB cells: (A) Prior to DIF-1 treatment, APP and ubiquitin are localized at discrete structures in the cell; and, (B) Upon DIF-1 treatment, APP and ubiquitin co-localize around the nuclear periphery.

### DETAILED DESCRIPTION OF THE INVENTION

The complex mechanisms that contribute to progressive neurodegenerative diseases such as Alzheimer's disease (AD) within the framework of neural development and cell cycle control suggest that in specific neuronal regions mitogenic pathways are activated early during the course of the disease. Although the majority of AD is late in onset, small portion of AD occurs earlier in life (e.g. <60 yr), and is caused by an inherited gene defect. The amyloid precursor protein (APP) is as an integral transmembrane protein that is widely expressed on the surface of cells and contains a 39-43 amino acid domain, termed Aβ, which is the main component of neuronal amyloid plaques (Haass et al., 1992). Although many signaling pathways are altered in AD, the consistent expression of cell cycle regulatory proteins (e.g., PCNA and cyclin D1) are detected in mild cognitive impairment (MCI) and AD afflicted brain regions which implies that irregular cell cycle events may idiopathically contribute to AD pathogenesis very early in the course of the disease (Nagy, 1999; Khurana et al., 2006; McShea et al., 1997; Busser et al., 1998; Hen⁻up and Arendt, 2002; Yang et al., 2006; Raina et al., 2000; Copani et al., 2001; Arendt, 2002; Bowser and Smith, 2002).

Differentiation-inducing factor-1 (DIF-1) (1-[3,5-dichloro-2,6-dihydroxy-4-methoxyphenyl]-1-hexanone) is a chlorinated alkyl phenone, isolated from the social amoeba Dictyostelium discoideum, that induces stalk cell differentiation during multicellular development (Kay and Jermyn, 1983; Morris et al., 1987). This molecule inhibits the growth of a number of cancer cell lines through novel, yet unknown signaling mechanism(s) that results in cell cycle arrest without inducing apoptosis (Asahi, 1995; Kubohara, 1999).

In an effort to identify novel molecules with both anti-tumor and anti-Aβ properties, the effect of DIF-1 was examined on the amyloidogenic processing of APP in Chinese Hamster Ovary (CHO) cells that stably overexpress human wild type APP⁷⁵¹ (CHO-7W), human APLP2 (CHO-A2) and human wild type APP⁷⁵¹/BACE1 (CHO-CAB) cells. In addition, the effect of DIF-1 on endogenous APP processing was also assessed in mouse embryonic fibroblasts (MEF) and human H4 neuroglioma cells that stably express the AD-associated Swedish mutant form of APP^{SW}. Results presented herein show that DIF-1 comparatively inhibited the proliferation of all cell types tested in a dose-dependent manner. The non-toxic, cytostatic effect of DIF-1 was attributed to a G0/G1 block of the cell cycle as examined by flow cytometry, and a concomitant decrease in cyclin Dl protein levels. DIF-1 decreased secreted APP, mature APP, APP C-terminal fragments and total Aβ levels. Notably, DIF-1 reduced the accumulation of both human wild type APP and wild type APLP2 C-terminal fragments (CTFs) alone or when cells were pretreated with the potent γ-secretase inhibitor DAPT, indicating that this enzyme was not affected by DIF treatment. Importantly, DIF-1 had no effect on human APLP1 CTF levels (CHO-A1) or the notch intracellular domain (NICD). DIF-1 reduced the level of total secreted APP, Aβ40 and Aβ42 in conditioned media collected from CHO-7W and CHO-CAB cells as measured by sandwich ELISA and western blotting. Phosphorylation of APP-CTF at residue T668 was reduced by DIF-1 in all cells tested. Pretreating cells with inhibitors of kinases implicated in the phosphorylation of APP at T668 (e.g. GSK3β, cyclin-dependent kinase 5 and c-Jun NH2-terminal kinase) did not prevent or potentiate the effect of DIF-1 on APP processing. This region of APP binds to Pin1 and may also interact with other class IV WW domain-containing proteins. CHO cells stably expressing forms of APP that cannot be phosphorylated at T668 (APPT668E or APPT668A) completely abolished the effects of DIF-1 on APP metabolism. These observations indicate that phosphorylation at this residue is linked to the mechanism by which DIF-1 alters APP metabolism. The data suggest that DIF-1 likely decreases amyloidogenic processing of APP through a novel phospho-dependent mechanism that requires the structural integrity of APP's C-terminal S/TP motif. Consistent with this notion, DIF-1 alters APP trafficking resulting in a peri-nuclear distribution of APP and an increased co-localization with the ER and ubiquitin. Finally, DIF-1 blocked the accumulation of APP C-terminal fragments and levels of APP ubiquitin-conjugates induced by the calpain inhibitor N-acetyl-leucyl-leucyl-norleucinal (ALLN). Taken together, these findings suggest that DIF-1 reduces amyloidogenic processing of APP and Aβ levels by enhanced targeting of APP to an ubiquitin-dependent ALLN-sensitive degradation pathway.

The invention is thus based at least in part on the surprising finding that Differentiation-inducing factor-1 (DIF-1) can inhibit γ-secretase-mediated proteolysis of APP and subsequent production of Aβ in a phosphorylation-dependent manner. Surprisingly, the inhibitory effect of DIF-1 on APP processing appears to be at least in part substrate-specific.

Thus, the invention provides novel uses of a small molecule (i.e., DIFs) that elicits inhibitory effects on the processing of APP. More specifically, the present invention is based on the discovery that DIF-1 affects γ-secretase-dependent proteolysis of APP. Based on the findings, the present invention describes various embodiments drawn to methods for inhibiting cellular production of toxic Aβ. Thus, the invention may be useful for treating and/or preventing diseases, neurodegenerative diseases in particular, which are associated with pathological accumulation of amyloid plaques and resulting dementia. The invention also embraces various formulations of therapeutic compositions, which comprise DIF-1 and/or its functional analogs.

### Differentiation-Inducing Factor-1 (DIF-1)

In recent years, much work has been focused on the identification of small molecules, both naturally occurring and synthetic, that exert biological activities that are useful for treating a wide of variety of diseases. One such candidate is the Differentiation-Inducing Factor-1 (DIF-1). DIF-1 is a chlorinated hexanophenone endogenously expressed in the eukaryote, Dictyostelium discoideum. While the exact molecular mechanisms are unknown, DIF-1, at very low concentrations, induces amoebae to differentiate into stalk cells and acts as a morphogen in the formation of the prestalk/prespore pattern during development.

Vegetative cells of Dictyostelium discoideum grow as single amoebae by eating bacteria, but when starved they start a developmental program of morphogenesis and gather to form a fruiting body consisting of spores and a multi-cellular stalk at the end of its development. Cyclic AMP (cAMP) is a well-known extra-cellular signal molecule essential for Dictyostelium development (Konijn T M, Van de Meene J G C, Bonner J T, Barkley D S. 1967. Proc Natl Acad Sci USA. 58:1152-4; Darmon M, Brachet P, Pereira da Silva L H. 1975. Proc Natl Acad Sci USA. 72:3163-6; Gerisch G, Fromm H, Husesgen A, Wick U. 1975. Nature 255:547-9).

The differentiation-inducing factors (DIFs) were originally identified as signal molecules that induce stalk-cell differentiation in vitro in the presence of cAMP (Town C D, Gross J D, Kay R R. 1976. Nature 262:717-9; Morris H R, Taylor G W, Masento M S, Jermyn K A, Kay R R. 1987. Nature 328:811-4; Kay R R, Berks M, Traynor D. 1989. Development, Suppl.:81-90).

The most active species, DIF-1, has been identified as 1-(3,5-dichloro-2,6-dihydroxy-4-methoxyphenyl)hexan-1-one (Morris H R, Taylor G W, Masento M S, Jermyn K A, Kay R R. 1987. Nature 328:811-4) and DIF-2, which is also referred to as DIF-1(−1), possessing ˜40% of the activity of DIF-1 as 1-(3,5-dichloro-2,6-dihydroxy-4-methoxyphenyl)pentan-1-one (Massento M S, Morris H R, Taylor G W, Johnson S J, Skapski A C, Kay R R. 1988. Biochem J. 256:23-8; Morris H R, Masento M S, Taylor G W, Jermyn K A, Kay R R. 1988. Biochem J. 249:903-6). DIF-3 [1-(3-chloro-2,6-dihydroxy-4-methoxyphenyl)hexan-1-one] is the initial product in the process of DIF-1 breakdown and is much less active in inducing stalk-cell differentiation (Kay R R, Berks M, Traynor D. 1989. Development, Suppl.:81-90; Morris H R, Masento M S, Taylor G W, Jermyn K A, Kay R R. 1988. Biochem J. 249:903-6; Kay R R, Flatman P, Thompson C R L. 1999. Semin Cell Dev Biol. 10:577-85). DIF-1 is thought to function, at least in part, via an increase in intracellular calcium concentration ([Ca2+]i), but the precise signaling system of DIF-1, including the target molecule(s) of DIF-1, is still unknown (Gokan et al., 2005. Biochemical Pharmacology 70: 676-685, reference incorporated herein).

DIF-1 (1-[3,5-dichloro-2,6-dihydroxy-4-methoxyphenyl]-1-hexanone) has a molecular mass of 306.0424, and an atomic composition of C₁₃H₁₆O₄Cl₂ The molecular structure of DIF-1 is shown below (c).

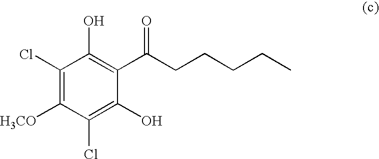

DIF-1 is soluble in both water and hexane at neutral pH, and it is therefore possible that it could penetrate cellular membranes.

Other DIF-1-like molecules have also been identified. For example, DIF-2 and DIF-3 are the pentanone and deschloro homologues of DIF-1, respectively. The molecular structure of DIF-2 (a) and DIF-3 (b) is provided below.

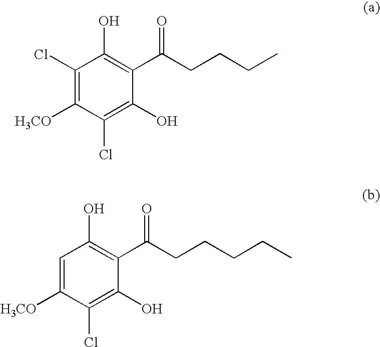

Like DIF-1, both DIF-2 and DIF-3 are endogenous (naturally occurring) differentiation-inducing factors, which induce stalk-cell differentiation in the cellular slime mould Dictyostelium discoideum. The absolute specific activity of DIF-1 in the context of stalk-cell differentiation has been reported to be in a sub-nanomolar range (Masento et al., 1988, Biochem J., 256:23-28). Relative biological activity of various DIF-1 analogs indicates that the C5 alkyl tail of DIF-1 represents the optimum chain length for bioactivity, although homologues differing by up to two methylene groups retained substantial activity. For example, studies conducted by Masento et al. showed that DIF-2, a naturally occurring C4 homologue, exerted ˜40% of the specific activity of DIF-1, while the C6 homologue had 14% activity. Similarly, in the same study, the brominated analogue was shown to possess ˜53% of the activity of DIF-1.

The biosynthesis pathways of these three factors in the organism have been partially elucidated. Two compounds are predicted to be intermediates in DIF-1 biosynthesis: the desmethyl, and desmethyl-monochloro analogs of DIF-1 (dM-DIF-1 and C1-THPH, respectively). The commonly used abbreviations include: DIF, Differentiation Inducing Factor, 1-(3,5-dichloro-2,6-dihydroxy-4-methoxyphenyl)hexan-1-one; dM-DIF-1,1-(3,5-dichloro-2,4,6-trihydroxyphenyl)hexan-1-one; C1-THPH, 1-(3-chloro-2,4,6-trihydroxyphenyl)hexan-1-one; and MPBD, 4-methyl-5-pentylbenzene-1,3-diol. The biosynthetic pathways and the endogenous enzymes that catalyze the biosynthesis of DIFs, as well as effects of mutations in the associated genes, have been identified and partially characterized. For review, see Williams, J. G. 2006, EMBO reports 7: 694-98.

The present invention embraces using DIF-1 as well as its analogs, salts thereof and/or solvates thereof for methods and compositions described herein. As used herein, “an analog” is a molecule or compound that is structurally and functionally related to its parent molecule or compound. Some DIF-1 analogs are naturally occurring, e.g., DIF-2 and DIF-3, while others are synthetically produced. DIF-1 and its analogs include but are not limited to (See Gokan et al., Biochem Pharmacol. 2005 Sep. 1;70(5):676-85): DIF-1, DIF-2, DIF-3, Br-DIF-1, Br-DIF-3, I-DIF-1, I-DIF-3, TM-DIF-1, DIF-1(−2), DIF-3(−2), DIF-1(−1), 3(−1), DIF-1(+1), DIF-3(+1), DIF-1(+2), DIF-3(+2), DIF-1(3M), DIF-3(3M), DIF-1(CP), DIF-3(CP), THPH, TH-DIF-1, TH-DIF-3, Ph-DIF-1, Ph-DIF-3, Et-DIF-1, Et-DIF-3, Bu-DIF-1, Bu-DIF-3, CP-DIF-1, CP-DIF-3. It should be understood that many other modifications of the compounds are possible, and the present invention is not intended to be limited to the specific compounds listed above.

Chemical synthesis of DIF-1 and related molecules (e.g., analogs) is well documented. For details, see, for example, Gokan et al., 2005, Biochemical Pharmacol., 70: 676-685 and Masento et al., 1988, Biochem J., 256:23-28. Briefly, the Hoesch reaction between 3,5-dihydroxyanisole and hexanonitrile, in the presence of ZnCl₂/HCl and H₂O, generates two products, one of which is an unchlorinated precursor to DIF-1 (DMPH). Subsequently, DIF-1 can be synthesized at high yield from the precursor, DMPH, by addition of SO₂Cl₂ and EtOH in the presence of CHCl₃ at room temperature. Those skilled in the art will be able to readily obtain the compounds used in the present invention. In addition, DIF-1 is commercially available from, for example, Affiniti Research Products (Mamhead, UK).

Some of the enzymes that catalyze the biosynthesis of the DIF molecules in Dictyostelium have also been identified. In addition, some of the naturally occurring intermediates, i.e., precursors, are also known.

More recently, DIF-1 has been shown to be involved in at least some aspects of transcriptional regulation in Dictyostelium that control pattern formation (for review, see Williams, J. G. 2006, EMBO reports 7: 694-98).

In other cell systems, it has become increasingly clear that DIF-1 exhibits anti-tumor activity in several types of mammalian tumor cells, via Go/G1 cell cycle arrest. This inhibitory effect of DIF-1 on proliferation appears to be mediated, at least in part, by promoting their differentiation. Evidence suggests that DIF-1 suppresses gene expression of cyclin D1 in a number of cell types, including murine and human leukemia tumor cells, rat pancreatic AR42J cells, vascular smooth muscle cells and gastric cancer cells. It has been postulated that DIF-1 may induce activation of GSK3β, causing decreases in β-catenin, and cyclin D1 protein levels.

For example, Mori J, et al. (Exp Cell Res. 2005 Nov. 1;310(2):426-33. Epub 2005 Sep. 8) described effects of DIFs on carcinoma cell lines. More specifically, the authors report that DIF-1 and DIF-3 inhibit proliferation and induce differentiation in oral squamous cell carcinoma cell lines NA and SAS. The article further shows that the DIF effects are mediated by cyclin D1 degradation. Evidence is provided to suggest that DIF-1 induces degradation of cyclin D1 through the GSK-3beta-mediated phosphorylation of Thr286.

Gokan et al. determined structural requirements of Dictyostelium differentiation-inducing factors for their stalk-cell-inducing activity in Dictyostelium cells and anti-proliferative activity in K562 human leukemic cells (Biochem Pharmacol. 2005 Sep. 1;70(5):676-85). In this study, the authors presented the chemical structure-effect relationship of DIF derivatives. Using 28 analogs of DIF-1 and DIF-3, the authors examined stalk-cell-inducing activity in Dictyostelium HM44 cells (mutant strain) and anti-proliferative activity in human leukemia K562 cells.

Kubohara et al. studied effects of differentiation-inducing factors of Dictyostelium discoideum on human leukemia K562 cells and found that DIF-3 is the most potent anti-leukemic agent (Eur J Pharmacol. 1999 Sep. 17;381(1):57-62). Based on previous reports that DIF-1 exhibits anti-tumor activity in mammalian cells, this study examined the effects of six DIF analogs on DNA synthesis, cell growth, erythroid differentiation, and cytosolic free calcium concentration ([Ca2+]i) in human leukemia K562 cells. The DIF analogs used here were DIF-1, DIF-2 (which has pentanone in place of hexanone), DIF-3 (dechlorinated form of DIF-1), 2-MIDIF-1 (2-methoxy isomer of DIF-1), DMPH (dechlorinated form of DIF-3), and THPH (4-hydroxy substitution of DMPH).

Taken together, these observations suggest that DIFs may exhibit strong anti-proliferative activities and occasionally induce cell differentiation in mammalian cells (Asahi K, Sakurai A, Takahashi N, Kubohara Y, Okamoto K, Tanaka Y. 1995. Biochem Biophys Res Commun. 208:1036-9; Kubohara Y, Saito Y, Tatemoto K. 1995. Federation Eur Biochem Soc Lett. 359:119-22; Kubohara Y, Kimura C, Tatemoto K. 1995. Dev Growth Differ. 37:711-6; Kubohara Y. 1997. Biochem Biophys Res Commun. 236:418-22; Kubohara Y. 1999. Eur J Pharmacol. 381:57-62; Kubohara Y, Hosaka K. 1999. Biochem Biophys Res Commun. 263:790-6; Miwa Y, Sasaguri T, Kosaka C, Taba Y, Ishida A, Abumiya T, et al. 2000. Circ Res. 86:68-75; Kanai M, Konda Y, Nakajima T, Izumi Y, Nanakin A, Kanda N, et al. 2003. Oncogene 22:548-54; Takahashi-Yanaga F, Taba Y, Miwa Y, Kubohara Y, Watanabe Y, Hirata M, et al. 2003. J Biol Chem. 278:9663-70) and that DIF-3 is a most potent anti-tumor agent among DIFs (Kubohara Y. 1999. Eur J Pharmacol. 381:57-62; Takahashi-Yanaga F, Taba Y, Miwa Y, Kubohara Y, Watanabe Y, Hirata M, et al. 2003. J Biol Chem. 278:9663-70). As to the mechanism of the actions of DIFs, it has been shown that: (1) DIFs increase [Ca2+]i in some tumor cells, (2) DIFs activate Akt/protein kinase B (PKB) in human leukemia K562 cells, (3) DIF-1 inactivates STAT3 in gastric cancer cells, and (4) DIF-1 inhibits the expression of cyclins D/E and the phosphorylation of retinoblastoma protein (pRb) in vascular smooth muscle cells and K562 cells. Quite recently, we have found that calmodulin (CaM)-dependent cyclic nucleotide phosphodiesterase (PDE1) is a pharmacological and specific target of DIFs (Shimizu K, Murata T, Tagawa T, Takahashi K, Ishikawa R, Abe Y, et al. 2004. Cancer Res. 64:2568-71). Yet, the mechanisms underlying the actions of DIFs in mammalian cells remain to be elucidated.

As described in more detail herein, the invention provides novel effects of DIFs on APP processing. Specifically, the invention is at least in part based on the notion that DIFs can alter the metabolic processing of APP thereby reducing the level of toxic Aβ accumulation. Notably, the effect of DIF appears to be substrate-specific, and parallels reduced phosphorylation of APP at Thr668. The phosphorylation of APP does not appear to involve the action of already known APP kinases, such as GSK3β and cdk5.

### Amyloid Precursor Protein (APP)

Biochemical pathways and the mechanisms of APP processing leading to Aβ production are at least partially elucidated. APP is a ubiquitously expressed type 1 membrane glycoprotein with multiple isoforms. The mammalian APP family contains three members: APP and the APP-like proteins, APLP1 and APLP2. In vivo functions of APP, APLP 1 and APLP2 remain poorly understood.

### APP Processing

APP undergoes alternative splicing to generate various transcripts that result in several isoforms that range from 365 to 770 amino acids. The three isoforms that are predominant in most tissues are APP695, APP751 and APP770, although the relative abundance of the three isoforms varies from tissue to tissue and in different cell types. For example, APP695 is the predominant neuronal isoform, while APP770 and APP751 are more common in peripheral and glial cells.

Following post-translational modifications, including glycosylation, the “mature” form of APP is an integral type I membrane glycoprotein that is trafficked through the constitutive secretory pathway. It has a large amino terminal extracellular/luminal domain and a short cytoplasmic tail. APP metabolism involves both amyloidogenic and non-amyloidogenic proteolytic pathways. The process of APP proteolysis involves step-wise events, in which the luminal domain is first processed by either (i) α-secretase (non-amyloidigenic pathway) to generate membrane-tethered α-C-terminal fragment (α-CTF), or (ii) β-secretase (amyloidogenic pathway) to generate membrane-bound β-C-terminal fragment (β-CTF). In the former pathway, α-secretase cleaves APP between residues Lys16 and Leu17, which essentially precludes the generation of intact Aβ in a subsequent event. Therefore, cleavage of APP by activation of α-secretase is a relatively major and ubiquitous pathway of APP metabolism in most cells and is apathogenic. In contrast, in the second pathway above, β-secretase cleaves APP to generate the amyloidogenic intermediate, β-CTF, which is a precursor to Aβ generation.

In the second step of APP processing, the resultant α-CTF or β-CTF is now cleaved by a multimeric protein complex termed γ-secretase, generating the fragments, p3 and Aβ, respectively. Of the two fragments, the former is non-amyloidogenic and the latter is amyloidogenic.

Thus, a prevailing view of the pathogenesis of AD is that accumulation of Aβ which results from the APP processing involving β-secretase and γ-secretase described above is the key feature of neuropathogenesis in the brain of the affected subject. As used herein, “pathogenesis” or “neuropathogenesis” of AD refers to the process of disease development generally characterized by the formation of senile plaque, or neurofibrillary tangles, neuronal dysfunction, microglial cell activation, neuronal death, and clinical dementia in affected patients. The pathological end product of APP processing, Aβ, is the primary component of the hallmark senile plaques found in the brains of AD patients. As used herein, “APP metabolism” includes both normal (apathogenic) process, and abnormal (pathogenic) process that is amyloidogenic, involving the trafficking and proteolytic processing of APP. The term “amyloidogenic” means that APP processing results in the generation of the toxic form of end product, i.e., Aβ.

Much effort has focused on the inhibition of Aβ production, with one strategy being to inhibit γ-secretase, an enzyme complex composed of presenilin, nicastrin, APH-1, and PEN-2 (for review, see Haass, 2004). The development of selective γ-secretase inhibitors has allowed pharmacological reduction in Aβ production (for review, see Harrison et al., 2004). γ-secretase is a member of the I-CLiP protease family and cleaves a number of additional intramembrane substrates, including CD-44, Erb4, E-cadherin, Notch, and the Notch ligands Delta and Jagged 2 (for review, see, for example, Wolfe and Kopan, 2004). One of the best characterized is the transmembrane protein Notch, which is cleaved to become the transcriptionally active Notch intracellular domain (Hartmann et al., 2001; Wolfe and Kopan, 2004). Inhibition of its production has been identified as a potential concern for γ-secretase inhibitor therapy, because high levels of γ-secretase inhibition affect B- and T-cell maturation and ileal goblet cell formation (Searfoss et al., 2003; Wong et al., 2004).

As used herein, “APP processing” refers to one or more steps of the sequential proteolytic events that cleave APP to smaller fragments. Accordingly, the term “APP processing” encompasses both normal, i.e., non-pathogenic, cellular metabolic events involving APP proteolysis and pathogenic events that lead to abnormal accumulation of Aβ. As such, the term “APP processing” is intended to refer to each of the cleavage steps as well as to the overall process involving β-secretase and α-secretase, which produce APP-C99 and APP-C83, as well as γ-secretase which produce Aβ and p3, respectively.

As used herein “reducing Aβ production” means that there is a decrease in the amount of Aβ produced by APP processing; alternatively, there may be a relative reduction in the ratio of amyloidogenic peptides to non-toxic peptides. In the latter case, the equilibrium of APP processing is shifted towards the production of non-toxic peptide fragments relative to toxic counterparts.

### Phosphorylation-Dependent Regulation

Proteolysis of polypeptides can be modulated either by regulating the protease enzyme activity or by specific post-translational modifications of the substrate. Phosphorylation is such a post-translational modification that is responsible for an increased heterogeneity of APP-CTF species. It has been shown that phosphorylation of APP on residue threonine 668 (T668) may play a role in APP metabolism. In AD brains, phosphorylated APP accumulates in large vesicular structures in afflicted hippocampal pyramidal neurons that co-stain with antibodies against endosome markers and BACE1. Western blot analysis reveals increased levels of T668 phosphorylated APP COOH-terminal fragments in hippocampal lysates from many AD, but not control subjects. The production of Aβ is significantly reduced when phosphorylation of T668 is either abolished by mutation or inhibited by T668 kinase inhibitors. Based on the observation that non-AD patients do not contain levels of phosphorylated APP in the brain compared to those afflicted with AD, the present invention contemplates a potential avenue for treatment of AD in the form of small molecule inhibitors of APP phosphorylation.

According to the invention, the regulation of APP processing involves phosphorylation of APP. As disclosed in more detail in Example 4, DIF-1 can exert its effect on APP processing by suppressing phosphorylation of APP. More specifically, the mechanism appears to involve an inhibition of the phosphorylation state of APP at threonine 668. While a number of protein kinases have been suggested to play a role in the regulation of APP, including GSK-3β, SAPKaNK, p38, MAPKs, PKC, the reduction in phosphorylated APP CTFs by DIF may involve an unknown kinase, possibly one not previously identified. Additionally, a possibility remains that the effect is rendered at least in part via regulation of serine/threonine (Ser/Thr) protein phosphatases (PPs), such as PP1, PP2A and PP2B.

### Substrate Specificity

Because the primary feature of Alzheimer's disease neuropathogenesis is the excessive build-up of Aβ, understanding the processing of the amyloid precursor protein (APP) has been a major focus in studies of AD neuropathogenesis. In particular, based on the fact that pathogenic Aβ is produced from APP via stepwise proteolysis by β-secretase and γ-secretase, these enzymes are the targets for a pharmacological intervention in an attempt to alter harmful APP processing. Indeed, γ-secretase inhibitors that are currently available can reduce Aβ levels and thus represents a potential treatment for AD. Nevertheless, while γ-secretase inhibitors can decrease Aβ production, they may also cause intolerable side effects owing to unwanted impairment of γ-secretase activity that is needed for normal cellular functions. For example, γ-secretase inhibitors also block the proteolysis of numerous other cellular substrates besides APP (e.g., APLP1 and Notch), and may cause severe side effects. To circumvent the risk, it would be thus desirable to develop γ-secretase inhibitors that can selectively inhibit the processing of APP, without affecting its activity towards other cellular substrates.

The present invention demonstrates that the DIF-1 is selective for APP, because the processing of other cellular targets for γ-secretase, such as Notch, is not affected. Thus, DIF-1 preferentially reduces γ-secretase-dependent proteolysis of APP. Furthermore, it has been surprisingly discovered that the inhibitory effect of DIFs on APP processing is reversible.

As used herein, the term “selective”, “selectively”, or “selectivity” refers to the ability of a subject molecule (e.g., protein, ligand, compound) to exert preferential affinity to or preferential activity towards a (or a subset of) particular substrate, receptor or other interacting molecule over others. In the context of pharmacological reagents, for example, a selective agent, e.g., a selective ligand, selective blocker, or selective enzyme inhibitor, exerts preferential target specificity.

### Therapeutic Applications

Therefore, the invention contemplates methods of treating or preventing AD in a subject having the disease or a subject at risk of developing the disease by administering a composition comprising DIF-1, an analog thereof, or combination thereof.

In some embodiments, a composition may include more than one species of such molecules.

To reduce Aβ production, the invention provides that DIF-1 or its analogs may be administered to a subject so as to contact a target cell. According to the invention, “a cell” is preferably a mammalian cell, a human cell in particular, which expresses APP and is capable of depositing proteolytic products of APP. In particular, a preferred target cell is a brain cell, e.g., neuronal and glial cell, that can produce toxic deposits of Aβ, characteristic of Alzheimer's disease.

As used herein, the terms “subject” and “animal” are intended to include humans and non-human animals. Preferred subjects include a human patient who has, is suspected of having, or has a family history of Alzheimer's disease. Other preferred subjects include subjects that are treatable with the compositions of the invention. This includes those who have, are suspected of having, or have a family history of a disease or disorder in which a reduction of Aβ levels is therapeutically or prophylactically beneficial. For example, a genetic predisposition (especially towards early onset AD) can arise from point mutations in one or more of a number of genes, including the APP, presenilin-1 and presenilin-2 genes. Also, subjects who are homozygous for the epsilon4 isoform of the apolipoprotein E gene are at greater risk of developing AD.

Administration of the DIF molecules, analogs, and other compositions described herein to mammals other than humans, e.g., for testing purposes or veterinary therapeutic purposes, is carried out under substantially the same conditions as described above.

Thus, a preferred subject is a human subject who has been diagnosed with Alzheimer's disease or who is at risk of developing Alzheimer's disease.

Likewise, in addition to being useful for treatment of Alzheimer's disease, the invention may also be useful for treating, preventing or delaying the onset of other conditions in which reduced production of Aβ in the brain is therapeutically or prophylactically beneficial. Such conditions include diseases in which Aβ is a causative factor or symptom, such as: Down's syndrome, multi-infarct dementia, dementia puglistica, cerebrovascular amyloidosis (Cerebral Amyloid Angiopathy), Hereditary Amyloidosis with Cerebral Hemorrhage of the Dutch Type (HCHWA-D), Familial British Dementia, vascular dementia, and inclusion body myositis, and homozygocity for the apolipoprotein E4 allele. Other conditions may be known to those of skill in the art.

The invention described herein thus provides various embodiments of methods by which Aβ production is reduced in a target cell. For example, Aβ production may be reduced by at least 10%, 20%, 30%, 40%, 50% or more.

An “effective amount” is a dosage of the therapeutic agent sufficient to provide a medically desirable result. An effective amount may also, for example, depend upon the degree to which an individual has abnormally increased levels of Aβ. It should be understood that the therapeutic agents of the invention are used to treat or prevent the disease (such as Alzheimer's disease), that is, they may be used prophylactically in subjects at risk of developing the disease (such as Alzheimer's disease). Thus, an effective amount is that amount which can lower the risk of, slow or perhaps prevent altogether the development of the disease (such as Alzheimer's disease).

The factors involved in determining an effective amount are well known to those of ordinary skill in the art and can be addressed with no more than routine experimentation. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

The therapeutically effective amount of a pharmacological agent of the invention is that amount effective to treat the disease. For example, in the case of Alzheimer's disease, a desired response is inhibiting the progression of Alzheimer's disease. This may involve only slowing the progression of Alzheimer's disease temporarily, although more preferably, it involves halting the progression of the Alzheimer's disease permanently. This can be monitored by routine diagnostic methods known to those of ordinary skill in the art. The desired response to treatment of Alzheimer's disease also can be delaying the onset or even preventing the onset of Alzheimer's disease.

In a subject affected by Aβ accumulation, i.e., amyloid plaques, or at risk of developing such a condition, an effective amount of an agent of DIF-1 and/or its analogs may be assessed by medical procedures which are routine in the art. In some embodiments the effective amount of DIF-1 and/or its analogs may be intended to partially block APP processing (i.e., sub-optimal doses). For any compound described herein a therapeutically effective amount can be initially determined from cell culture assays. In particular, the effective amount of DIF-1 and/or its analogs can be determined using in vitro assays. Effective amounts of a composition of the invention may also be determined by assessing physiological effects of administration on a cell or subject, such as a level of Aβ that is released and relative ratio of toxic and non-toxic proteolytic products of APP following administration. Other assays will be known to one of ordinary skill in the art and can be employed for measuring the level of the response to a treatment.

The amount of a treatment administered may be varied for example by increasing or decreasing the amount of a therapeutic composition administered, by changing the therapeutic composition administered, by changing the route of administration, by changing the dosage timing and so on. The effective amount may vary with the particular condition being treated, the age and physical condition of the subject being treated, the severity of the condition, the duration of the treatment, the nature of the concurrent therapy (if any), the specific route of administration, and the like factors within the knowledge and expertise of the health practitioner. For example, an effective amount may depend upon the stage and pathogenesis of the disease. These factors are well known to those of ordinary skill in the art and can be addressed with no more than routine experimentation.

Therapeutically effective amounts can also be determined in animal studies. For instance, the effective amount of DIF-1 and/or its analogs alone or in combination with other therapy to induce a therapeutic response can be assessed using in vivo assays, such as behavioral assessment, as well as histological analyses, such as brain immunohistochemistry and plaque formation. Relevant animal models include mouse models of AD. Generally, a range of DIF-1 and/or its analogs doses are administered to the animal. Reduction in the level of the toxic Aβ species produced in the animals following the administration of DIF-1 and/or its analogs is indicative of the ability to reduce the APP processing and thus treat or prevent AD.

In a patient, clearance of Aβ from the brain following administration of the relevant compounds may be evidenced by an increase in the level of soluble Aβ in the cerebrospinal fluid and/or the plasma. Alternatively (or additionally), imaging techniques such as magnetic resonance imaging, positron emission tomography, single photon emission computed tomography and multiphoton microscopy may be employed to monitor the extent of Aβ deposition in the brain (see, for example, Bacskai et al., J. Cereb. Blood Flow Metab., 22 (2002), 1035-41).

The patient's degree of cognitive decline or impairment is advantageously assessed at regular intervals before, during and/or after a course of treatment in accordance with the invention, so that changes therein may be detected, e.g., the slowing or halting of cognitive decline. A variety of neuropsychological tests are known in the art for this purpose, such as the Mini-Mental State Examination (MMSE) with norms adjusted for age and education (Folstein et al., J. Psych. Res., 12 (1975), 196-198, Anthony et al., Psychological Med, 12 (1982), 397408; Cockrell et al., Psychopharmacology, 24 (1988), 689-692; Crum et al., J. Am. Med. Assoc'n. 18 (1993), 2386-2391). The MMSE is a brief, quantitative measure of cognitive status in adults. It can be used to screen for cognitive decline or impairment, to estimate the severity of cognitive decline or impairment at a given point in time, to follow the course of cognitive changes in an individual over time, and to document an individual's response to treatment. Another suitable test is the Alzheimer Disease Assessment Scale (ADAS), in particular the cognitive element thereof (ADAS-cog) (See Rosen et al., Am. J Psychiatry, 141 (1984), 1356-64).

In certain embodiments, additional treatments for Alzheimer's disease can be used in conjunction with the methods of the invention. Conventional AD treatments include, but are not limited to: cholinesterase inhibitors, including donepezil (Aricept®), rivastigmine (Exelon®), galantamine (Remix®), and tacrine (Cognex®); NMDA receptor antagonists including memantine (Namenda®); AMPA receptor agonists including CX516 (Ampalex®); choline uptake enhancers, including MKC-231; and HMG CoA reductase inhibitors, i.e., statins. Other treatments include immune therapy.

The applied dose of DIF-1 and/or its analogs (and optionally other therapy) can be adjusted based on the relative bioavailability and potency of the administered compounds. The pharmacological agents used in the methods of the invention are preferably sterile and contain an effective amount of DIF-1 and/or its analogs for producing the desired response in a unit of weight or volume suitable for administration to a subject. The doses of pharmacological agents administered to a subject can be chosen in accordance with different parameters, in particular in accordance with the mode of administration used and the state of the subject. Other factors include the desired period of treatment. Adjusting the dose to achieve maximal efficacy based on the methods described herein and other methods are well within the capabilities of the ordinarily skilled artisan. These amounts can be adjusted when they are combined with DIF-1 and/or its analogs by routine experimentation. For example, in the event that a response in a subject is insufficient at the initial doses applied, higher doses (or effectively higher doses by a different, more localized delivery route) may be employed to the extent that patient tolerance permits.

Subject doses of the compounds described herein typically range from about 0.1 μg to 10,000 mg, more typically from about 1 μg/day to 1000 mg, and most typically from about 10 μg to 100 μg. Stated in terms of subject body weight, typical dosages range from about 0.1 μg to 20 mg/kg/day, more typically from about 1 to 10 mg/kg/day, and most typically from about 1 to 5 mg/kg/day.

It is generally preferred that a maximum dose of an composition to effectively reduce undesirable Aβ production but less than the dose that would significantly inhibit (or otherwise affect) other cellular targets of γ-secretase (alone or in combination with other therapeutic agents) be used, that is, the highest safe dose according to sound medical judgment. It will be understood by those of ordinary skill in the art, however, that a patient may insist upon a lower dose or tolerable dose for medical reasons, psychological reasons or for virtually any other reasons.

In some cases, it may be desirable to target the pharmaceutical compound to a specific tissue or tissues. For example, many compounds have poor bioavailability to the brain, which is a site of the Aβ plaque accumulation in neurodegenerative diseases such as AD. Co-administration with hydroxyurea may promote the availability of the pharmaceutical compound to the brain.

Generally, a pharmaceutical compound may be coupled to a vectorizing agent to facilitate bioavailability in a desired tissue or tissues. As used herein, the term “vectorized” refers to engineered moieties or modifications to a subject agent or compound for the purpose of delivering the composition to a target site in a cell or a tissue. For example, vectorized agents are produced by covalently linking a compound to a moiety which promotes delivery from the circulation to a predetermined destination in the body. Accordingly, the present invention further contemplates agents of DIF-1 and its analogs that are “directed” to the brain of a subject by the means of vectorized agents. In some examples, antibodies are linked to another macromolecule, the antibodies being the agent which promotes delivery of the macromolecules. One example of such an agent is an antibody which is directed towards a cell surface component, such as a receptor, which is transported away from the cell surface.

In some embodiments of the invention, DIF-1 and/or DIF-1 analogs may be administered along with one or more additional pharmaceutical compounds. In these cases, the other therapy may be administered at the same time or in alternating cycles or any other therapeutically effective schedule. “Alternating cycles” as used herein, refers to the administration of the different active agents at different time points. The administration of the different active agents may overlap in time or may be temporally distinct. The cycles may encompass periods of time which are identical or which differ in length. For instance, the cycles may involve administration of the DIF-1 on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc, with the other therapy being administered in between. Alternatively, the cycles may involve administration of DIF-1 on a daily basis for the first week, followed by a monthly basis for several months, and then every three months after that, with the other therapy being administered in between. Any particular combination would be covered by the cycle schedule as long as it is determined that the appropriate schedule involves administration on a certain day.

Various modes of administration will be known to one of ordinary skill in the art which effectively deliver DIF-1 and/or its analogs to reduce Aβ production in a cell. Methods for administering such a composition, or other pharmaceutical compound of the invention may be intravenous, oral, intracranial, intrathecal, intracavity, intrathecal, intrasynovial, buccal, sublingual, intranasal, transdermal, intravitreal, subcutaneous, intraperitoneal, subcutaneous, intramuscular or intradermal administration. The invention is not limited by the particular modes of administration disclosed herein. Standard references in the art (e.g., Remington's Pharmaceutical Sciences, 20th edition, 2000) provide modes of administration and formulations for delivery of various pharmaceutical preparations and formulations in pharmaceutical carriers. Other protocols which are useful for the administration of therapeutic compound of the invention will be known to one of ordinary skill in the art, in which the dose amount, schedule of administration, sites of administration, mode of administration and the like vary from those presented herein.

The active compounds can be prepared with carriers that will protect the compound against rapid release, such as a controlled release formulation, including implants, transdermal patches, and microencapsulated delivery systems. DIF-1 and/or its analogs described in the present invention that are useful in the treatment of AD and other diseases that are associated with toxic accumulation of amyloid plaques may be conjugated to or in association with a delivery vehicle such as a nanocarrier. Examples of nanocarriers include, but are not limited to, liposomes, immunolipososomes, microparticles, emulsions, etc. These and other suitable delivery vehicles and methods of their use are known to those of ordinary skill in the art.

Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The compositions may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The formulations provided herein also include those that are sterile. Sterilization processes or techniques as used herein include aseptic techniques such as one or more filtration (0.45 or 0.22 micron filters) steps.

The methods provided herein include compositions comprising DIF-1, an analog or combination thereof, optionally formulated with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with additional AD or related drug formulations in a manner such that there is no interaction which would substantially impair the desired pharmaceutical efficacy.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the active agent may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrants include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the active agent either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

In some embodiments, DIF-1 and/or its analogs may be delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park (N.C., USA); and the Spinhaler powder inhaler, manufactured by Fisons Corp. (Bedford, Mass., USA).

All such devices require the use of formulations suitable for the dispensing of the compound of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated.

Formulations suitable for use with a nebulizer, either jet or ultrasonic, will typically comprise the compound of the invention dissolved in water at a concentration of about 0.1 to 25 mg of biologically active compound per mL of solution. The formulation may also include a buffer and a simple sugar. The nebulizer formulation may also contain a surfactant, to reduce or prevent surface induced aggregation of the active agent caused by atomization of the solution in forming the aerosol.

Formulations for use with a metered-dose inhaler device will generally comprise a finely divided powder containing the active agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof Suitable surfactants include sorbitan trioleate and soya lecithin. Oleic acid may also be useful as a surfactant.

Formulations for dispensing from a powder inhaler device will comprise a finely divided dry powder containing active agent and may also include a bulking agent, such as lactose, sorbitol, sucrose, or mannitol in amounts which facilitate dispersal of the powder from the device, e.g., 50 to 90% by weight of the formulation. The active agent should most advantageously be prepared in particulate form with an average particle size of less than 10 μm (or microns), most preferably 0.5 to 5 μm, for most effective delivery to the distal lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The invention will be more fully understood by reference to the following examples. These examples, however, are merely intended to illustrate the embodiments of the invention and are not to be construed to limit the scope of the invention.

### Examples

The following examples further illustrate the invention but are not intended to limiting its scope in any way.

### Introduction

### Metabolic Processing of APP

Alzheimer's disease (AD) is the major form of dementia that affects the elderly in aging populations and can be broadly characterized by an early loss in memory combined with extensive cognitive deterioration. It is a slow, progressive neurodegenerative disorder defined pathologically by increased extracellular deposits of amyloid (plaques) and the formation of intracellular neurofibrillary tangles (NFT) in specific neuronal regions of the brain. The principle cellular and molecular mechanisms of the disease are unknown. There is no adequate treatment and there are no reliable diagnostic methods that can accurately identify AD-related prognostics before the onset of severe clinical symptoms. However, small sections of the population have an inheritable genetic component (at least 3 genes) that accelerates the onset of disease (Hardy et al., 1998; Schellenberg et al., 1995; Selkoe, 1997; Tanzi et al., 1994). Presenilin 1 (PS1) is the major gene responsible for FAD, and point mutations found throughout the PS1/PS2 proteins lead to early onset FAD (Sherrington et al., 1995; Rogaev et al 1995; Levy-Lahad et al., 1995). Mutations in the amyloid β protein precursor protein gene located on chromosome 21 are also responsible for a small percentage of early-onset cases of AD (EOAD) (Chartier-Harlin et al., 1991; Goate et al., 1991; Levy et al., 1990; Mullan et al., 1992; Murrell et al., 1991). It has also been shown that duplication of the APP locus may also lead to EOAD, but without the clinical features seen in Down's syndrome (Cabrejo et al., 2006).

The amyloid β-protein precursor protein is highly conserved type I transmembrane glycoprotein that is subject to a variety of post-translational modifications, and is expressed in most cells, but is enriched in neurons (Haass et al., 1992). The discovery of APP led to the identification of two mammalian homologues, amyloid precursor-like proteins 1 (APLP1) and APLP2 (Wasco et al., 1992; Wasco et al., 1993). Although this family of proteins exhibits a high degree of structural similarity, only APP contains the toxic 38 to 43 amino acid Aβ domain, which represents the main component of AD-associated amyloid plaques (Walsh and Selkoe, 1994). A number of functional roles for APP have been suggested, yet their physiological significance and how the precise regulation of APP processing contributes to AD pathology remain unknown. However, most of the familial (FAD)-associated mutations found in APP reside within close proximity to the Aβ domain, and in some manner modulate the enzymatic production of Aβ42/Aβ42 (Xia et al., 1997). As such, accumulating evidence has led to the proposed amyloid cascade hypothesis that implicates altered APP processing and toxic Aβ aggregation as the dominant pathogenic factor in AD (Walsh and Selkoe, 1994). APP is processed by at least two different mechanisms in multiple signaling pathways which results in the production of different carboxy-terminal cleavage fragments (CTFs). Cleavage of APP by either α- or β-secretases produces the soluble N-terminal fragments sαAPP and sβAPP, and smaller C83 and C99 membrane-bound CTFs, respectively. Each of these CTF molecules can be further cleaved by γ-secretase leading to the release of either the non-pathogenic p3 peptide or Aβ (Haass et al., 1992; Shoji et al., 1992). The non-amyloidogenic cleavage of APP initiated by α-secretase(s) appears to be the dominant metabolic pathway in most cells (Nitsch et al., 1992). The amyloidogenic pathway is initiated by cleavage of APP by the β-secretase (BACE1) and produces the 99 amino acid β-CTF. Subsequent processing by γ-secretase results in the generation of Aβ40 and to a lesser extent Aβ42. The identification of the enzymes primarily responsible for APP processing has been paramount in not only elucidating the physiological significance of APP metabolism, but sets the stage for the exploitation of both β- and γ-secretases as pharmacological targets for the prevention or treatment of AD. The most obvious benefit to fully understanding the normal cellular signaling mechanisms that regulate APP metabolism is the development of methods for early detection, identification of target(s) for drug intervention, and ultimately the prevention of AD.

### AD and the Cell Cycle Connection

Although the amyloid cascade hypothesis dominates the AD research landscape, a number of alternative theories have been suggested in attempts to understand the pathogenesis of AD. These hypotheses include tau phosphorylation, inflammation, oxidative stress, metal ion dysregulation and Ca²⁺-dyshomeostasis (Nagy et al., 1998; Zhu et al., 2000). Many lines of experimental evidence support the idea that each of these mechanisms can contribute to AD pathogenicity, yet no single theory can account for all of the observed neurological changes that occur throughout the disease process. Recent studies have provided ample evidence to suggest that irregular cell cycle re-entry within neuronal populations may play an early, yet crucial role for abnormalities associated with AD pathogenesis (Raina et al., 2000; Copani et al., 2001; Arendt, 2002; Herrup and Arendt, 2002). Neuronal central dogma states that fully differentiated neurons are post-mitotically locked, and thus cannot cycle or divide. Regulation and protection from neuronal cell cycle re-entry is achieved through strict cell cycle control checkpoints. Yet the plasticity of neurons and the extension of neurites involve progression of the cell cycle early in G1 phase, followed by strict signaling mechanisms for neuronal re-differentiation back into a differentiated state of GO without further consequences. Such transient re-entry into the cell cycle is believed to be an integral part of synaptic remodeling (Arendt, 2003). However, in AD, the G1/S check-point mechanisms appear to deteriorate to the point where individual neurons can proceed through S-phase, resulting in the replication of DNA and entry into the G2 phase of the cell cycle (Yang et al., 2001; Lopez et al., 2005). In normal cycling non-neuronal cells, the G2 phase is critical to prepare the cell for mitotic division, but in the case of post-mitotic neurons, aberrant cycling invariably stops during G2, and the neurons have no choice but to die by way of apoptosis. Recent evidence coming from a number of animal models of human neurodegenerative diseases supports the hypothesis that atypical cell cycle re-entry precedes neuronal apoptosis leading to neuronal death (Yang et al., 2006; Khurana et al., 2006). Increased levels of many cell cycle proteins, including PCNA, cyclin D1, CDK4, and cyclin B1, have been detected in the hippocampus, and other AD-diseased brain regions. Cell cycle markers are not detected randomly throughout the diseased brain nor are they found in age-matched control-patient brains (McShea et al., 1997; Busser et al., 1998; Herrup and Arendt, 2002). In support of this, early neurological studies suggested activated cell cycles occur only during neurogenesis or in neoplastic neurons. What is of more importance is that cell cycle markers are not only one of the earliest neuronal abnormalities detected in AD, but can theoretically lead to most if not all of the observed pathological conditions associated with the disease including tau phosphorylation, Aβ formation and neuronal ion dysfunction (Vincent et al., 1998; Zhu et al., 2000; Khurana et al., 2006). Data on cell cycle activation has also been reported in other neurological diseases such as ALS (Amyotrophic lateral sclerosis), Parkinson's and Huntington diseases, ataxias, stroke and trauma (Khurana et al., 2006). The ectopic expression of cell cycle markers has been documented in at least four different strains of APP-transgenic mice including the well-known Tg2576 mouse, months before amyloid deposition could be detected (Yang et al., 2006). Breakdown of cell cycle checkpoint mechanisms can be attributed to either inheritable defects or the result of cumulative environmental insults. Mutations found in cell cycle regulatory proteins in either neurons or non-neuronal cell types significantly increases the risk of developing diseased cell types. Of particular interest with respect to APP function are the reports that pancreatic cancer cells, oral squamous cell carcinomas and colon carcinomas all display an increase in the expression and processing of APP suggesting a role for APP in cellular proliferation (Hoffman et al., 2000; Schmitz et el, 2002; Osahwa et al., 1999; Siemes et al., 2006, Kummer et al., 2002; Caine et al., 2004; Hayashi et al., 1994; Meng et al., 2001). Research has shown that ADAM10 α-secretase activity releases the secreted form of sαAPP, and that ADAM10 mRNA and protein levels are upregulated 2-fold in squamous cell carcinomas (Ko, et al., 2004; Ko et al., 2007). Indeed many different kinds of signaling pathways are changed in AD, and the relevance of the mitogenic up-regulation that may induce cell cycle re-entry in the disease process is far from clear.

### Dictyostelium and the Role of DIF-1 During Development

The social amoeba Dictyostelium discoideum is an excellent model organism for the analysis of many basic cellular processes such as differentiation, development and motility. Basic biological research in Dictyostelium has without a doubt enhanced our understanding of higher eukaryotic biology. Dictyostelium primarily grows as an independent amoeboid cell. The multicellular stage is activated in response to starvation (Kessin, 2001). It is at this time where up to 10⁵ cells collectively chemoattract to secreted cAMP and converge to form a mound of cells. Cells within the mound are organized by specific cell-cell interactions and the release of small molecular morphogens that signal their coordinated differentiation into prestalk or prespore cell types (Thompson et al., 2004; Williams et al., 1989). The cells within the mound are oriented and migrate into specific regions to form a slug where prestalk and prespore cells organize along the anteroposterior axis. The final stage of development relies upon the controlled secretion of morphogens resulting in the terminal differentiation of prestalk and prespore cells. At this point, a true multicellular fruiting body has formed, that embodies a ball of spores supported by dead stalk cells (Kessin, 2001). Attempts to elucidate the essential molecular and cellular mechanisms required to complete such a highly synchronized pattern-forming process, resulted in the discovery of a unique chlorinated alkyl phenone (Kay, 1983). This small molecule, (1-[3,5-dichloro-2,6-dihydroxy-4-methoxyphenyl]-1-hexanone) termed differentiation factor-1 (DIF-1) was found to be crucial for multicellular development in this organism (Kay et al., 1983). DIF-1 was identified as a diffusible molecule released by developing Dictyostelium cells that induces differentiation of amoeba into stalk cells (Morris et al., 1987). The exact signaling role of DIF-1 during Dictyostelium development has yet to be fully elucidated. However, a consensus has now emerged that posits that DIF-1 synthesis occurs in prespore cells (Kay and Thompson, 2001) and regulates the normal differentiation of the pstO cell population during development (Kimmel and Firtel, 2004; Strmecki et al., 2005).

### Evidence of DIF-1 Mediated Effects in Mammalian Cells

The molecular and cellular effects of DIF-1 are not limited to Diclyostelium, but also elicit specific effects on mammalian cells. DIF-1 has a strong inhibitory effect on the proliferation of tumor cell lines yet the mechanism(s) and molecules responsible for this effect are not well understood (Asahi et al., 1995; Kubohara, 1999; Kanai et al., 2003; Yasmin et al., 2005). Furthermore, mammalian analogs of DIF-1 have yet to be found. It has also been shown that furanodictines (FDs), aminosugar analogs found in D. discoideum, can lead to the neuronal differentiation of PC12 cells (Kikuchi et al., 2006). DIF-1 induces transient activation of glycogen synthase kinase-3β (GSK3β) in an in vitro kinase assay, which leads to a decrease in cyclin D1 protein and mRNA levels implicating DIF-1as a modulator of the Wnt/β-catenin signaling pathway (Yasmin et al., 2005). However, in that study GSK3β activity was assessed by the detection of Ser-9 phosphorylation over a period of only three hours. The effect of DIF-1 on GSK3β was greatest after one hour, but was comparably negligible after three hours when compared to controls. A reduction in cyclin D1 expression is critical for cell cycle progression into S-phase, such that it serves as a marker of cell cycle progression and a regulator of cell cycle machinery preventing uncontrolled proliferation (Matsushime et al., 1994). Phosphorylation of cyclin D1 at Thr-286 has been suggested as one mechanism for its degradation. Although the exact identity of the cyclin D1 Thr-286 kinase is uncertain, in vitro evidence suggests that the constitutively active kinase GSK3β phosphorylates Thr-286 thereby targeting cyclin D1 for rapid proteasomal degradation (Diehl et al., 1997). However, unlike cyclin D1, the level and activity of GSK3β remains relatively unchanged throughout the cell cycle and may not be the only kinase involved in regulating degradation of cyclin D1 levels during S phase (Yang et al., 2006). The use of small molecule inhibitors, LiCl, GSK3β siRNA and expression of a constitutively active mutant GSK3β all failed to alter cyclin D1 levels in human and murine fibroblast cells (Yang et al., 2006). Cyclin D1 protein levels are also closely linked to stabilized cytosolic β-catenin levels via Wnt signaling pathways. Cytosolic β-catenin forms complexes with the Tcf-Lef transcription factor and translocates into the nucleus, where it activates transcription of specific cell cycle genes that regulate proliferation (Morin, 1999). The deregulation of β-catenin signaling is thus an important event in the genesis of a number of malignancies, such as colon cancer, melanomas and prostrate cancer (Morin, 1999). Interestingly, β-catenin has been identified as one of many presenilin-interacting proteins implicated as a modulator of Wnt signaling (Zhou et al., 1997). The physiological functions of presenilins are relatively still unknown, but they may be related to developmental signaling and apoptotic signal transduction. The presenilins are also essential components of the γ-secretase enzymatic complex responsible for amyloidogenic cleavage of APP (De Strooper et al., 1998: Wolfe et al., 1999). Curiously, PS1 deficient mice accumulate cytosolic β-catenin, display an increase in Tcf/Lef-dependent cyclin D1 transcription, and contribute to the development of skin tumors (Xia et al., 2001). Tumor cells, unlike normal cells, typically exhibit increased proliferation through uncontrolled progression through the cell cycle. The highly potent anti-tumor properties of DIF-1 in mammalian cells suggests this molecule may target specific Wnt signaling proteins that have been implicated in a variety of human cancers as well as neurodegenerative diseases, specifically that of AD. Yet, the precise mode of DIF-1 action, and the target molecule(s) it may modulate are unknown in both mammalian cells and Dictyostelium. Thus, Dictyostelium could serve as a legitimate resource in contributing relevant lead compounds that may be exploited pharmacologically with a considerable impact in translational biomedical research.

In an effort to identify novel molecules with both anti-tumor and anti-Aβ properties, we examined the effect of DIF-1 on cell-cycle dependent amyloidogenic processing of APP. We assessed the dose-dependent effect of DIF-1 on cell proliferation and APP processing in a variety of cell cultures including those that stably express human wild type APP⁷⁵¹, the Swedish mutant form of APP^{Sw} and APP⁷⁵¹/BACE1. Here, we show that when actively growing cultured cells are pharmacologically treated with DIF-1 (1-30 μM) the number of cells found in G0/G1 increases as assessed by flow cytometry. In keeping with this, western blots show that cyclin D1 levels are consistently decreased by ˜82.56±0.74% compared to controls. Similar amounts of DIF-1 irreversibly inhibit the proliferation of CHO, CHO-7W's, CHO-CAB, mouse neuroblastoma N2a and human SH—SY5Y lines. The effect of DIF-1 thus appears to be cytostatic. Further to this, the processing of APP CTFs was drastically reduced by DIF-1 in all cell types tested. The levels of human wild type APLP2 CTFs were also reduced by DIF-1 in CHO cells stably expressing APLP2 (CHO-A2). Importantly, DIF-1 had no effect on the CTF levels of human wild type APLP1 (CHO-A1) or the notch intracellular domain (NICD). Notably, DIF-1 treatments caused modest reductions in APP⁷⁵′ maturation. However, significant reductions in total APP and Aβ secretion were detected in conditioned media. Interestingly, DIF-1 was shown to reduce Aβ42 (66.9±2.6%) more so than the levels of Aα40 (44.2±3.4%), respectively. Furthermore, the phosphorylation of APP⁷⁵¹ at residue T668 (APP⁶⁹⁵ numbering) is significantly reduced (86%±5.6) by DIF-1 in all cells tested. Sequence analysis of APP suggests that the S/TP region encompassing T668 is a potential phosphorylation-dependent interaction motif specifically recognized by class IV WW domain proteins including the peptidyl-prolyl cis/trans isomerase Pin1 (Pastorino et al., 2006). Cells pretreated with an array of inhibitors for kinases implicated in the phosphorylation of T668 including GSK3β (LiCl, kenpaullone, 1-azakenpaullone), cdk5 (roscovitine) and the c-Jun NH2-terminal kinase (SP600125) neither prevented nor potentiate the observed effect of DIF-1 on APP processing. Surprisingly, CHO cells stably expressing a mutant form of human APP⁷⁵¹ that cannot be phosphorylated at residue T668 (APPT668E or APPT668A) each abolished the entire effect of DIF-1 on APP processing. Our findings suggest that DIF-1 may decrease the level of APP maturation, metabolism and secretion of Aβ through a novel phospho-dependent mechanism which requires the intrinsic structure of the S/TP motif neighboring T668. The proposed mechanism for this novel compound with respect to APP processing and differential reduction of Aβ42 species is discussed.

### Methods

### Reagents and Antibodies

DIF-1 (1-(3,5-dichloro-2,6-dihydroxy-4-methoxyphenyl)-1-hexanone) was purchased from Affiniti Research Products (BioMol®, United Kingdom). Polyclonal anti-BACE1 C-terminus rabbit (PA1-757) was purchased from Affinity BioReagents™ (Colorado, USA). Polyclonal anti-BACE pro-domain (N-term 26-45) was purchased from BioSource International, Inc.™ (California, USA). Antibodies to phosphorylated APP (αAPPT668p), polyclonal anti-caspase-3 antibodies were purchased from Cell Signaling Technology® (Massachusetts, USA). Anti-APP C-terminal polyclonal antibodies (8717), Anti-β-catenin rabbit (C2206) and anti-PS1-Nterm (P7854), lysosomal marker Anti-LAMP2 antibody (L0668), Hoechst 33462 nuclear stain, DIF-3 were purchased from Sigma-Aldrich® (Missouri, USA). Rabbit anti-notch1 (NICD) polyclonal (AB5709), monoclonal anti-BACE C-terminus (61-3E7) antibody, mouse monoclonal anti-GAPDH antibodies (MAB374), monoclonal N-terminal APP antibodies (22C11) were purchased from Chemicon, International, Inc. (California, USA). Pre-cast 14% and 8% Tris-glycine gels, SeeBlue® plus pre-stained MW markers, Alexa Fluor rabbit secondary antibodies (A-11034) and mouse secondary antibodies (A-11032) were purchased from Invitrogen™ Corporation (California USA). Polyclonal cyclin D1 (H-295) antibody, phospho-GSK3β (ser9) were purchased from Santa Cruz Biotechnology®, Inc. (California, USA). Anti-APP antibodies to amino acid residues 1-17 of Aβ (6E10), plasma membrane marker pan-Cadherin monoclonal antibody (ab6528), ER marker mouse anti-PDI monoclonal antibody (ab12225), golgi marker anti-GM130 monoclonal antibody (ab1299) were purchased from AbCam® PLC (United Kingdom). The γ-secretase inhibitor IX (DAPT), GSK3β inhibitors kenpaullone and 1-azakenpaullone, the cdk inhibitor roscovitine, lysosomal inhibitors chloroquine and ammonium chloride, proteasome inhibitors MG-132, ALLN or lactacystin, anti-GSK3α/β monoclonal antibodies (1H8) were purchased from Calbiochem®. Polyclonal antibodies to APP C-terminus (C66) were kindly provided by Dr. Dora Kovacs (Massachusetts General Hospital). Polyclonal anti-APP N-term (an 595-611) (1736) was kindly provided by Dr. Jack Rogers (Massachusetts General Hospital) (R1736) (Haass C, et al. 1992). Polyclonal sAPPβ antibodies were kindly provided by Dr. Rudy Tanzi (Massachusetts General Hospital). Polyclonal APLP1-W1Ct antibody and polyclonal APLP2-W2Ct antibody were provided by Dr. W. Wasco and Dr. D. Walsh (Massachusetts General Hospital). Lithium Chloride was purchased from Fisher Scientific™ (Massachusetts, USA). PVDF membranes and filter pads were purchased from BioRad®, Laboratories, Inc. (California, USA). BCA protein assay reagent and SuperSignal™ ECL™ were purchased from Pierce Biotechnology (Illinois, USA).

### Cell Culture

Naïve CHO, N2a, MEF and SY5Y cells were maintained in Dulbecco's modified Eagle's medium containing 4.5 mg/ml D-glucose supplemented with 10% fetal bovine serum and 100 units/ml penicillin, 100 μg/ml streptomycin, 2 mM L-glutamine (Sigma) at 37° C. in a 5% CO₂ atmosphere. CHO cells stably expressing human wild type APP⁷⁵¹ (CHO-7W) (Koo and Squazzo, 1994), wild type human APLP2 or wild type human APLP1 (courtesy of Dr. D. Walsh) were grown in media supplemented with hygromycin (Invitrogen). CHO cells stably expressing human APP⁷⁵¹ and human BACE1 (CAB) were maintained in G418 and zeocin. H4 neuroglioma cells stably expressing human wild type APP⁷⁵¹ or the Swedish mutant form APP^{SW} were maintained in G418.

### Cell Proliferation Assays

For proliferation assays, 1×10⁴ cells were deposited into 24-well plates and treated with vehicle (ethanol or DMSO) or with various amounts of DIF-1 or DIF-3 for a period of 4 days. Cells were harvested after each day by trypsin/EDTA treatment, stained with an equal volume of 0.05% Trypan blue and enumerated in triplicate twice using a haemocytometer. Dead cells are stained blue, but live cells with intact cell membranes are not coloured. Data represents the mean cell number±the standard deviation for at least 3 separate experiments.

### Flow Cytometry

Naïve and CHO-7W cells were collected by trypsin/EDTA treatment and counted using a haemocytometer. The cells (1×10⁵) were deposited into 10 cm tissue culture dishes containing fresh media and grown for 24 hours prior to DIF-1 treatment (25 μM). Cells were grown in the presence of DIF-1 for approximately 20 hours. Cells harvested by the trypsin/EDTA treatment were suspended in hypotonic fluorochrome solution containing 50 ug/ml of propidium iodide, 0.1% sodium citrate, and 0.1% Triton X-100. Cells (5×10⁴) for each sample were analyzed for fluorescence using the Massachusetts General Hospital cytology core. Cell cycle distribution was compared between vehicle and DIF-1 treated nave CHO cells, and CHO-7W cells. The percentage of cells in G0/G1, S-phase and G2/M represents the mean cell number±the standard deviation from at least 3 separate experiments.

### Construction of APPT668A and APPT668E Mutation, Stable Cell Lines

To assess whether or not the decrease in phosphorylated APP seen in DIF-1 treated cells contributed mechanistically to the reduction of maturation, secretion and metabolic processing of APP⁷⁵¹, mutants were generated using APP⁷⁵¹ in which T724 (APP⁷⁵¹ numbering) was mutated to either an alanine residue such that phosphorylation could not occur, or a glutamic acid residue that should mimic constitutive phosphorylation of APP. APP⁷⁵¹, Isoform B, NM_{—}201413 was used as a template. We essentially used Stratagene's QuickChange® Site-Directed Mutagenesis Kit (Catalog #200518) to create the desired mutations as recommended by the manufacturer with the following exceptions. The APP⁷⁵¹ codon for threonine 724 (ACC) was mutated to code for Alanine (A) (GCC) and Glutamic Acid (E) (GAA), respectively. The following mutagenesis primers were used to create each mutation. For the T724A mutation, the forward primer consisted of 5′-GACGCCGCTGTCGCCCCAGAGGAGCGC-3′ [SEQ ID NO:1] and the reverse primer 5′-GCGCTCCTCTGGGGCGACAGCGGCGTC-3′ [SEQ ID NO:2]. For the T724E mutation, the forward primer consisted of 5′-GTTGACGCCGCTGTCGAACCAGAGGAGCGCCAC-3′, [SEQ ID NO:3] and the reverse primer 5′-GTGGCGCTCCTCTGGTTCGACAGCGGCGTCAAC -3′ [SEQ ID NO:4]. The mutated codons for each mutation are underlined. The following PCR conditions were used with 50 ng of template DNA: one denaturing cycle for 30 seconds at 95° C., followed by 12 cycles consisting of 30 seconds at 95° C., 1 minute at 57.4 or 55.3° C., and 12 minutes at 68° C. The PCR products were transformed into E. coli using Invitrogen's MAX Efficiency™ DH5-α™ Competent Cells (Catalog #18258-012) and plated on LB agar with containing ampicillin to select for the ampicillin-resistant clones. Colonies were subsequently selected and grown in LB media supplemented with ampicillin for ˜16 hours at 37 degrees. Plasmids were purified using Qiagen's (Valencia, Calif., USA) QIAPrep® Spin Miniprep Kit (Catalog #27104) and sequenced at the Massachusetts General Hospital's Sequencing Core to identify positive or negative mutants using the following sequencing primers: T724 (forward) 5′-ATCTCCAGCCGTGGCATT-3′ [SEQ ID NO:5] and T724 (reverse) 5′-TTCGTAGCCGTTCTGCTG-3′ [SEQ ID NO:6]. All primers were purchased from Operon™ Biotechnologies, Inc. (Alabama, USA). Naïve CHO cells were transfected with 2 μg of plasmid DNA using the AMAXA Nucleofector® kit optimized for CHO cells (amaxa Inc., Gaithersburg, Md., USA).

Cells were diluted and plated in multiple 10 cm tissue culture dishes and allowed to recover overnight in Dulbecco's modified Eagle's medium containing 4.5 mg/ml D-glucose supplemented with 10% fetal bovine serum and 100 units/ml penicillin, 100 μg/ml streptomycin, 2 mM L-glutamine (Sigma) at 37° C. in a 5% CO₂ atmosphere. The media was changed the next day and supplemented with G418 at a final concentration of 600 μg/mL for a period of 3 weeks. The media was changed every 2 days to maintain the levels of G418. After 3 weeks, single colonies were carefully picked to 96-well dishes with 0.5 ml media containing G418 in each well and grown to confluency. Cells were then replica plated using 24-well tissue culture dishes and dilution plated in 10 cm dishes media containing G418. Once the cells in a 24-well dish reached confluency, media was removed; the cells were washed twice with warm PBS and lysed in cold RIPA buffer. Protein extracts were assessed for APPT724A and APPT724E expression levels compared to naïve cells by western blot using polyclonal anti-APP C-terminal antibodies (C66). However, for clarity purposes we will refer to these mutations as APPT668A and APPT668E (APP⁶⁹⁵ numbering).

### Protein Extraction, Western Blotting and Immunoprecipitation

Following treatment of each cell line with or without DIF-1 (1-30 μM) and/or other inhibitors, cells were lysed in cold RIPA buffer (20 mM Tris-HCl (pH 7.4) buffer containing 150 mM NaCl, 2 mM EDTA, 1% Nonidet® P-40 (NP-40), 50 mM NaF, 1 mM Na₃VO₄, 1 mM Na₂MoO₄, 10 mg/ml aprotinin, and 10 mg/ml leupeptin), supplemented with protease inhibitor tablets from Roche (Switzerland). Protein amounts were quantified in triplicate using the BCA assay from Pierce. Equal amounts of protein extracts were fractionated by sodium dodecyl sulfate polyacrylamide gel electrophoresis (SDS-PAGE) and transferred to polyvinylidene difluoride membranes. The membranes were blocked for 1 hour with 5% fat-free powdered milk or 5% BSA, incubated overnight at 4° C. with primary antibodies (1:1000 or 1:5000), washed with TTBS and then incubated at room temperature with appropriate peroxidase-conjugated secondary antibodies (1:5000). Blots were repeatedly washed with TTBS and target proteins were detected using an enhanced chemiluminescence (ECL) system (Amersham Pharmacia Biotech, New Jersey, USA) and XOMAT™ Kodak® film.

For immunoprecipitations, 1 mg of total protein was diluted with extraction buffer and precleared with protein G-conjugated agarose beads (Pierce). The precleared samples were incubated overnight with anti-APP antibodies or nonimmune IgG (Jackson Laboratories). Immunocomplexes were captured with Protein G agarose beads, washed five times with extraction buffer and heated for 5 min at 95° C. in SDS gel-loading buffer.

### Detection and Quantification of Total Soluble APP and Secreted Aβ in Conditioned Media

The levels of secreted Aβ from CHO-7W cells stably overexpressing APP⁷⁵¹ and CHO-CAB cells that overexpress APP⁷⁵¹ and human BACE1 was quantified by ELISA and viewed by western blot using the monoclonal anti-APP antibody (6E10) that recognizes residues 1-17 of Aβ. Cells (5×10⁵) were deposited into 6-well tissue culture dishes in triplicate and allowed to settle and grow for 24 hours. At this time, the media was aspirated and the cells were washed 2× in warm PBS. Fresh media (2 mL) and media supplemented with vehicle or DIF-1 was added to each well. After 20 hours, conditioned media was collected from untreated, vehicle and DIF-1 (25 μM) treated cells, and centrifuged at 14,000 rpm for 10 minutes to remove any cells or debris. Since DIF-1 exerts a cytostatic effect, the cell number was counted for all treatments upon removal of the conditioned media and used to normalize the levels of Aβ. It should be noted that after 20 hours, the total cell number for vehicle treated cells is ˜10% higher than DIF-1 (25 μM) treated cells. The concentration of Aβ40 and Aβ42 was subsequently detected in triplicate using the β-Amyloid 1-40 or 1-42 Colorimetric ELISA kit (Biosource International, Inc.) according to the manufacturer's instructions.

### Densitometry and Statistical Analysis

Densitometry analysis of western blot data was performed on a PC computer using QuantityOne® software obtained from Bio-Rad® Laboratories, Inc. (California, USA). Loading variations between western blot lanes were normalized according to the GAPDH signal prior to any experimental quantification. The number of samples (n) in each experimental condition is indicated in figure legends. Each western blot presented in the figures is representative of at least five independent experiments to ensure reproducibility of the results unless noted otherwise. Statistical analysis was performed using Instat3 software. An unpaired t test was employed for data sets that passed a normality test. An unpaired t test with Welch correction was employed for data sets that passed a normality test but had different standard deviations.

### Confocal Fluorescence Microscopy

CHO-CAB cells were grown on multi-well chamber slides and treated overnight with 30 μM DIF-1. The cells were fixed with 4% paraformaldehyde/PBS, permeabilized with 0.1% Triton X-100/PBS for 10 min and blocked with 5% goat serum/PBS. APP (rabbit C-terminal 8717), PDI (ER marker), GM130 (golgi marker), and ubiquitin were detected using their corresponding antibodies and Alexa Fluor-conjugated secondary antibodies. Nuclei were stained with Hoechst 33342 (Molecular Probes/Invitrogen). Confocal fluorescence images were obtained using an Olympus DSU/IX70 spinning disc confocal microscope and processed using IPLab software (Scanalytics/BD Biosciences).

### Results

### DIF-1 Reduces the Proliferation of CHO Cells and CHO-7W Cells Stably Expressing APP⁷⁵¹ by Inducing G0/G1 Cell Cycle Arrest and Reduced Expression of Cyclin D1

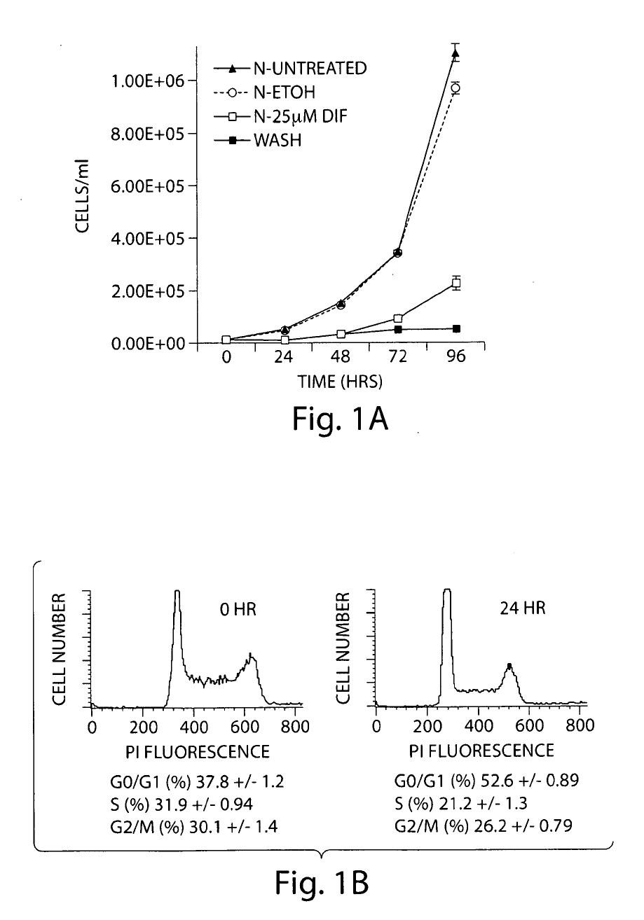

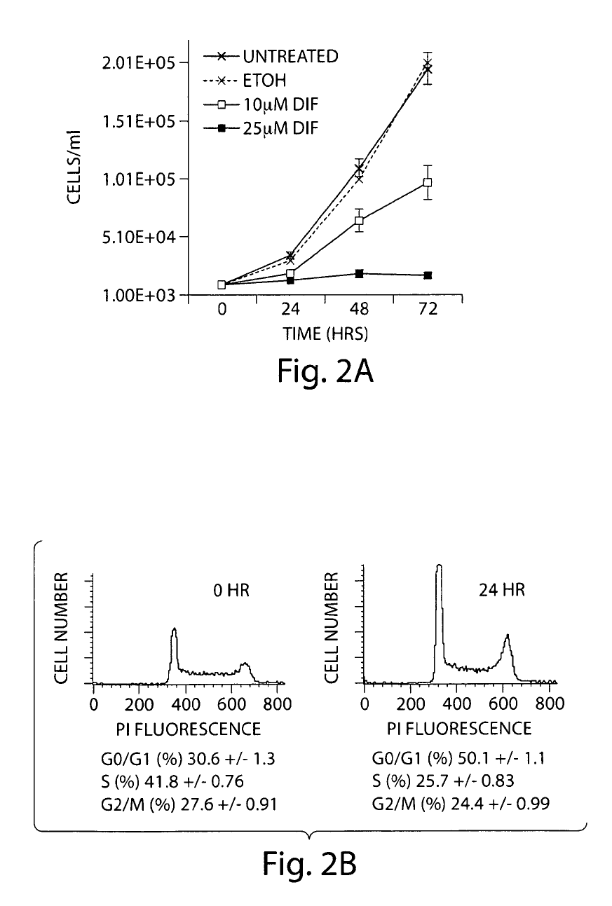

Although it has been shown that DIF-1 exhibits a strong in vitro inhibitory effect on the proliferation of mammalian tumor cell lines (Kubohara, Y. 1999; Kanai et al., 2003; Yasmin et al., 2005), the antiproliferative effect has not been comparatively assessed in normal cell lines. We therefore assessed the dose-dependent anti-proliferative effect of DIF-1 on normal (non-transformed) CHO cells (FIG. 1A). Briefly, CHO cells were seeded on a 6-well plate (10,000 cells/well) and were incubated with 25 μM DIF-1. Cells were harvested by the trypsin/EDTA treatment at the times indicated and counted with a haemocytometer. FIG. 1A shows the time course of the increase in cell numbers (Results shown are the means+/−s.e.m. from three independent experiments (n=3 for each experiment)). As demonstrated, cells treated for 24 hours with DIF-1 at 25 μM showed a minor, yet insignificant inhibition of growth (FIG. 1A). However, after 72 hours all cell lines tested (e.g. CHO, N2a and SH-SY5Y) exhibited a 70-75% decrease in proliferation compared to control and vehicle treated cells (FIG. 2). Therefore, for this study we only analyzed the effect of DIF-1 between 18 and 20 hours. It should be noted that the duration of DIF-1 treatment (or vehicle) had no observable effect on cell viability (trypan blue exclusion) in any of the cell lines tested, and its antiproliferative effect was readily reversed by simply washing treated cells with PBS and replacing it with fresh growth media (FIG. 1A). These findings suggest that the duration of treatment and the concentrations DIF-1 (30 μM) is non-toxic, but rather it imparts a cytostatic property that leads to growth arrest in non-transformed cell lines. Furthermore, DIF-1 did not effect cell adhesion, cell shape or induce apoptosis as measured by caspase-3 activation and western blotting using anti-caspase 3 polyclonal antibodies.

To determine where in the cell cycle DIF-1 acts, we assessed the cell cycle distribution of naïve CHO cells using flow cytometry (FIG. 1B). CHO cells were incubated with DIF-1 (25 μM) for the indicated periods and then harvested by the trypsin/EDTA treatment, fixed and stained with propidium iodide (PI) and fluorescence of nuclei was measured. Histogram peaks are labeled by phases (G0/G1, S and G2/M). The percentages of cell number in the cell cycle phases are shown as means+/−S.E. of three independent experiments performed in duplicate. The percentage of non-treated or vehicle treated cells found to be in the G0/G1 phase was on average 37.8%±1.2. However, cells grown overnight in media containing 25 μM DIF-1 consistently increased the number of cells in G0/G1 (52.6%±0.9) and reduced the percentage of cells in either S or G2/M phases (FIG. 1B). Thus DIF-1 appears to inhibit cell cycle progression in the G0/G1. This data confirms earlier findings that DIF-1 inhibits the proliferation of tumor cells (Kubohara, Y. 1999; Kanai et al., 2003; Yasmin et al., 2005), but also shows that DIF-1 elicits a strong cytostatic effect on a number of different mammalian cell lines regardless of origin.

Since roles in cellular proliferation have been suggested for both APP and APLP2, in epithelial cells, keratinocytes and neural stem cells (Hoffman et al., 2000; Schmitz et al, 2002; Osahwa et al., 1999; Siemes et al., 2006, Kummer C. et al., 2002; Caine et al., 2004; Hayashi et al., 1994) we next assessed the dose-dependent effect of DIF-1 on CHO cells stably expressing human wild type APP⁷⁵¹ (CHO-7W). Cells seeded on a 6-well plate were incubated with various concentrations of DIF-1. Cells were harvested by the trypsin/EDTA treatment at the times indicated and counted with a haemocytometer. The time course of the increase in cell numbers is shown in FIG. 2A. Results shown are the means+/−s.e.m. from three independent experiments (n=3 for each experiment). Overexpression of APP⁷⁵¹ did not inhibit or accelerate the antiproliferative effect of DIF-1 when compared to naïve cells (30.6%±1.3) (FIG. 2A).

Flow cytometry analyses were carried out to assess cell cycle distribution, as shown in FIG. 2B. CHO/APP cells were incubated with DIF-1 (25 μM) for the indicated periods and then harvested, fixed and stained with propidium iodide (PI) and fluorescence of nuclei was measured. Histogram peaks are labeled by phases. The percentages of cell number in the cell cycle phases are shown as means+/−S.E. of three independent experiments performed in duplicate. The effects of DIF-1 on CHO-7W cell growth is also a result of G0/G1 cell cycle block (50.1%±1.1) as measured by flow cytometry (FIG. 2B). To our knowledge, no proliferative role has yet to be suggested for APLP1.

We also analyzed the ability of DIF-1 to inhibit proliferation of neuronally-derived mouse N2a and human SH-SY5Y cells. Cyclin D1 is synthesized early in G1 phase and plays a key role in the initiation and progression of this phase and its reduction in expression is critical for progression into S-phase, thus it serves as an excellent marker of cell cycle phase and progression (Matsushime et al., 1994). Because it has been reported that DIF-1 may induce G0/G1 arrest through the suppression of D-type cyclins in tumor cells (Kanai et al., 2003), and that increased levels of cyclin D1 have been detected AD-diseased brain regions using immunohistochemistry (Busser et al., 1998) we assessed the effect of DIF-1 on cyclin D1 expression in our system.

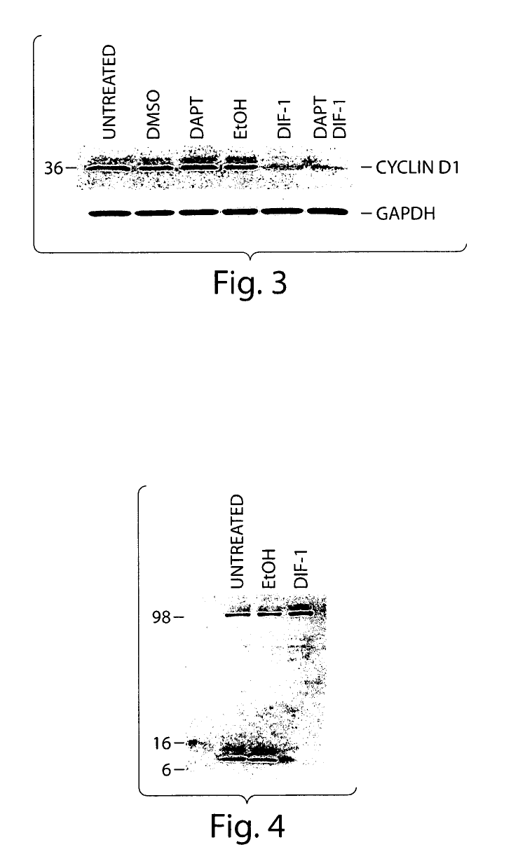

CHO/APP cells were treated for 24 hours with 30 μM DIF-1, 250 nM DAPT or DIF-1 and DAPT together. 15 μg of total soluble protein was analyzed by western blot using polyclonal anti-cyclin D1 antibodies (H-295). A representative result is shown in FIG. 3. Whereas DAPT had no effect on cyclin D1 levels, the level of cyclin D1 in CHO-7W cells was markedly decreased when treated 18-20 hours with 30 μM DIF-1 (FIG. 3). The level of cyclin D1 was also considerably reduced in both CHO-CAB cells and mouse embryonic fibroblasts when treated 18-20 hours with 30 μM DIF-1 (an 82.56±0.74% reduction). Similar results were obtained using CHO-CAB, MEF and H4 neuroglioma cells. It therefore appears that the cytostatic effect of DIF-1 is due to a mechanism that is correlated with the degradation of cyclin D1 and G0/G1 cell cycle arrest in a variety of different mammalian cell lines tested thus far.

### The Effect of DIF-1 on Metabolic Processing of APP, APLPs and Aβ Production

We next assessed the effect of DIF-1 on APP metabolism in CHO-7W cells by Western blotting analyses (shown in FIG. 4). Briefly, CHO-7W cells were treated for 24 hours with 30 μM concentrations of DIF-1. Total protein was collected in RIPA buffer plus protease inhibitors on ice and briefly sonicated. They lysate was centrifuged at 14,000 rpm for 15 minutes. 15 μg of total soluble protein was analyzed by western blot using polyclonal anti-APP Cterminal antibodies (1:5000) (C66, courtesy of Dr. D. Kovacs). After washing, the blots were probed with a polyclonal HRP-tagged secondary Ab (1:5000) for 1 hour at room temperature then visualized. Cells treated overnight with 30 μM DIF-1 resulted in a significant decrease in APP maturation and both αCTF and βCTF production (FIG. 4).

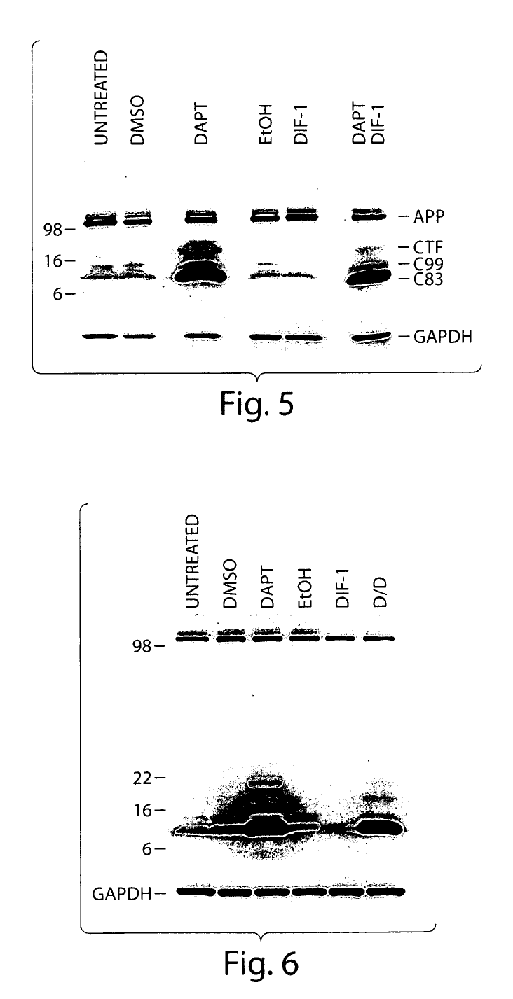

The findings suggest a number of possibilities, one being that processing of APP by α-secretases and the β-secretase (BACE1) in CHO cells are regulated by the phase of the cell cycle. A second possibility is that DIF-1 potentiates the activity of y-secretase and processing of αCTFs and βCTFs. To assess this latter possibility, we examined the effect of DIF-1 in the presence of DAPT, a potent γ-secretase inhibitor. Even numbers of CHO-7W cells were plated in 6-well dishes and allowed to grow overnight. The next day, the media was removed and replaced with control media (2 ml) or media containing DAPT, DIF-1 or DAPT and DIF-1. Cells were treated for ˜20 hours with 30 μM DIF-1, 250 nM DAPT or DIF-1 and DAPT together. 15 μg of total soluble protein was analyzed by western blot using polyclonal anti-APP C-terminal antibodies (C66) (1:5000) overnight at 4° C. After washing, blots were probed with HRP-conjugated secondary antibodies (1:5000) for 1 hour at room temperature. As shown in FIG. 5, the ˜22 kDa band is present only when cells are treated with DAPT. However, the intensity of the band is greatly reduced by the presence of DIF-1. The results show that DAPT treatment of CHO-7W cells results in an increase in the levels of both α- and βCTFs as expected, but cells treated with DIF-1 alone caused significant decreases in both αCTF and βCTF levels (FIG. 5). When CHO-7W cells were simultaneously treated with DAPT and DIF-1, the levels of both αCTF and βCTF were increased, but not to the level seen with DAPT alone as assessed by western blotting (FIG. 5). The presence of DIF-1 still exhibited a reduction in each CTF, but the effect was more evident for βCTFs (FIG. 5). It would appear that the mechanism of DIF-1 on APP metabolism is not likely to involve γ-secretase activity, but might impact on either BACE1 levels or activity.

To evaluate this probability, we treated CHO-CAB cells that overexpress human APP⁷⁵¹ and human BACE1 with 30 μM of DIF-1. More specifically, even numbers of CHO-CAB cells were plated in 6-well dishes and allowed to grow overnight. The next day, the media was removed and replaced with control media (2 ml) or media containing DAPT, DIF-1 or DAPT and DIF-1. Cells were treated for ˜20 hours with 30 μM DIF-1, 250 nM DAPT or DIF-1 and DAPT together. 15 μg of total soluble protein was analyzed by western blot using polyclonal anti-APP C-terminal antibodies (C66) (1:5000) overnight at 4° C. After washing, blots were probed with HRP-conjugated secondary antibodies (1:5000) for 1 hour at room temperature. Each treatment is shown above the blot. Interestingly, the effect on the amount of detectable mature APP after DIF-1 treatments varies slightly from the CHO-7W cells shown in FIG. 5. It would appear that DIF-1 has a greater effect on APP maturation in CHO-CAB cells. Longer exposures reveal the presence of mature APP bands in DIF-1 treated cells.

The results indicate that Overexpression of BACE1 did not repress the effects of DIF-1 on APP metabolism (FIG. 6). To ascertain if DIF-1 indiscriminately perturbs the metabolism of type I transmembrane proteins we investigated the processing of other γ-secretase substrates. Co-treatment of CHO cells stably expressing human APLP2 with DIF-1 and DAPT also resulted in a decrease in APLP2 CTF levels (FIG. 6B).

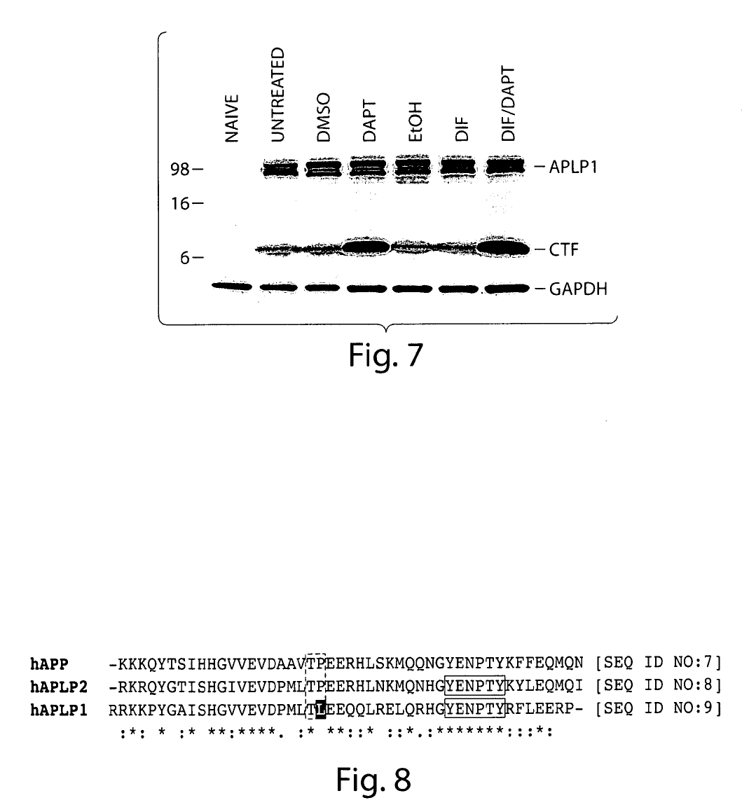

CHO cells that stably express human APLP1, which is a closely related family member of the APPs and known γ-secretase substrate (Eggert et al., 2004), were treated with 30 μM DIF-1, DAPT or DIF-1 plus DAPT (FIG. 7). Equal numbers of CHO-A1 cells stably expressing human APLP1 cells were plated in 6-well dishes and allowed to grow overnight. The next day, the media was removed and replaced with control media (2 ml) or media containing DAPT, DIF-1 or DAPT and DIF-1. Cells were treated for ˜20 hours with 30 μM DIF-1, 250 nM DAPT or DIF-1 and DAPT together. 15 μg of total soluble protein was analyzed by western blot using polyclonal anti-APLP1 C-terminal antibodies (W1Ct) (1:1000) overnight at 4° C. After washing, blots were probed with HRP-conjugated secondary antibodies (1:5000) for 1 hour at room temperature. Of interest, DIF-1, at 30 μM had no effect on APLP1 maturation, protein levels or CTF production with or without DAPT.

As expected, treatments using only DAPT caused a significant increase in the levels of APLP1 CTF. Importantly, DIF-1 had no effect on APLP1 maturation or CTF levels. Therefore, it would appear that the action of DIF-1 on APP and APLP2 may involve a shared signaling mechanism and is not due to an unspecific effect on the metabolism of all type I transmembrane proteins. In order to gain some insight as to how DIF-1 reduces APP and APLP2 CTF levels, but not that of the highly similar APLP1, we aligned the last 99 amino acids of each protein and looked for potential structural similarities and/or differences.

As depicted in FIG. 8, the alignment between the last 47 residues of APP [SEQ ID NO:7] and the APLPs [SEQ ID NO:8 & SEQ ID NO:9] reveals the presence of significant similarity between this family of type 1 transmembrane proteins. The highly conserved YENPTY motif [SEQ ID NO:10] believed to affect APP trafficking and internalization through its interaction with adapter proteins (e.g., Fe65) is highlighted in medium gray in FIG. 8. Another region of interest predicted to be a proline-directed class IV WW phosphorylation-dependent interaction motif is highlighted in light gray (S/TP). Interestingly, this type of protein interaction motif is not found in APLP1 due to the lack of a proline residue critical for the function of this type of motif. APLP1 contains a leucine (L) residue following the threonine (T) and is highlighted in dark gray. The name of each protein is shown on the left. Identical residues between all three C-terminal domains are denoted with an asterisk (*), where as homologous residues are denoted with a colon (:) below the alignment. This family of proteins has maintained a high level of conservation not only in humans, but across many vertebrate species as well (Coulson et al., 2000). The cytoplasmic domain contains a clathrin internalization sorting signal (NPXY) [SEQ ID NO:11] and at least eight putative phosphorylation sites that may play a role in APP metabolism (Suzuki et al., 1994: Selkoe et al., 1996; Oishi et al., 1997; Standen et al., 2001; Tan et al., 2002; Lee et al., 2003; Walsh et al., 2007). Thus, an alignment between APP and the APLPs reveals that APP and APLP2 each contain a proline-directed class IV WW phosphorylation-dependent interaction motif (S/TP) that is not found in APLP1 (FIG. 8). This predicted domain contains the threonine residue 668.

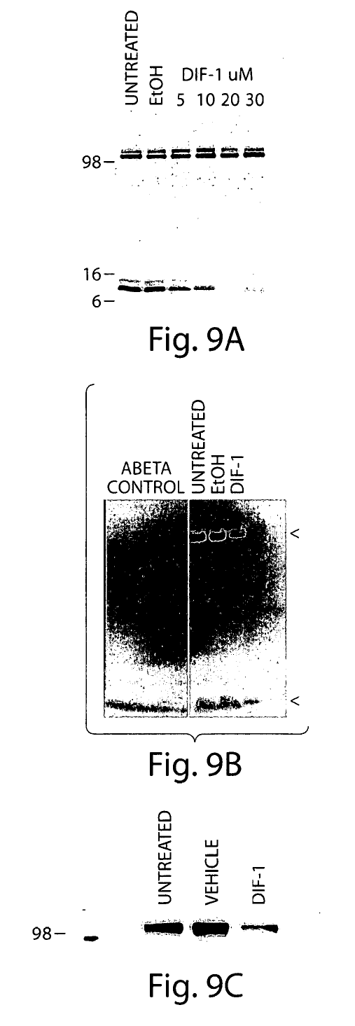

Next dose-dependent effects of DIF-1 on APP processing were examined. Even numbers of CHO-CAB cells were plated in 6-well dishes and allowed to grow overnight. The next day, cells were treated overnight with either vehicle or increasing concentrations of DIF-1 (5-30 μM). 15 μg of total soluble protein was analyzed by western blot using polyclonal anti-APP C-terminal antibodies (1:5000) (C66). After washing, the blots were probed using an HRP-conjugated secondary antibody (1:5000) for 1 hour at room temperature. A representative result of these experiments are provided in FIG. 9A. As the dose of DIF-1 increased, the amount of αCTF and βCTF decreased. Furthermore, it appears that ˜20 μM of DIF-1 was required to have an effect on mature APP levels. Thus, DIF-1 reduced both αCTFs and βCTFs in a dose-dependent manner (FIG. 9A).

We then looked at what effect this had on the levels of secreted soluble APP and Aβ production. Cells were deposited (1×10⁶) into 6-well tissue culture flasks and allowed to grow overnight. The next day, the media was aspirated, and fresh media supplemented with increasing concentrations of DIF-1 (1-30 μM) or vehicle was added and incubated for 18 to 20 hours. At that time, conditioned media and total cell protein extracts were collected from DIF-1 treated, non-treated and vehicle treated cells (CHO-7W and CHO-CAB) and transferred to sterile microcentrifuge tubes, respectively. Replica plates were also set-up and used to count the number of cells in each well post-treatment. Since DIF-1 is cytostatic, we needed to normalize our Aβ sandwich ELISA data to both cell number and total protein collected between samples. For these experiments, conditioned media was collected from equal numbers of CHO-7W non-treated, vehicle-treated and DIF-1 (30 μM) treated cells. The conditioned media was analyzed using anti-APP antibody 6E10 in order to detect total βP levels and sαAPP levels since it recognizes the amino acids 1-17 of Aβ and thus cannot detect sβAPP. Control amounts of Aβ were also loaded and blotted to the same membrane and are shown on the left. The blot was cut in half prior to probing with anti-APP antibody 6E10 in case the Aβ controls produced too high of a signal. The blots were analyzed using a phosphorimager. DIF-1 reduced the level of total secreted APP detected in the conditioned media and also reduced the level of total Aβ detected by western blot using anti-APβ 6E10 polyclonal antibodies (FIG. 9B).

To analyze total secreted soluble APP from CHO-CAB cells treated with 30 μM DIF-1, conditioned media was collected from CHO-CAB cells treated overnight with vehicle or 30 μM DIF-1 and then analyzed by western blotting for total sAPP levels using anti-APP antibody 22C11 (FIG. 9C). The blot shows that DIF-1 appears to have a greater effect on DIF-1 metabolism involving β-cleavage of APP as the levels of sAPP are much lower than seen in FIG. 9B. This is also supported by DIF-l′s greater reduction in βCTF levels and Aβ.

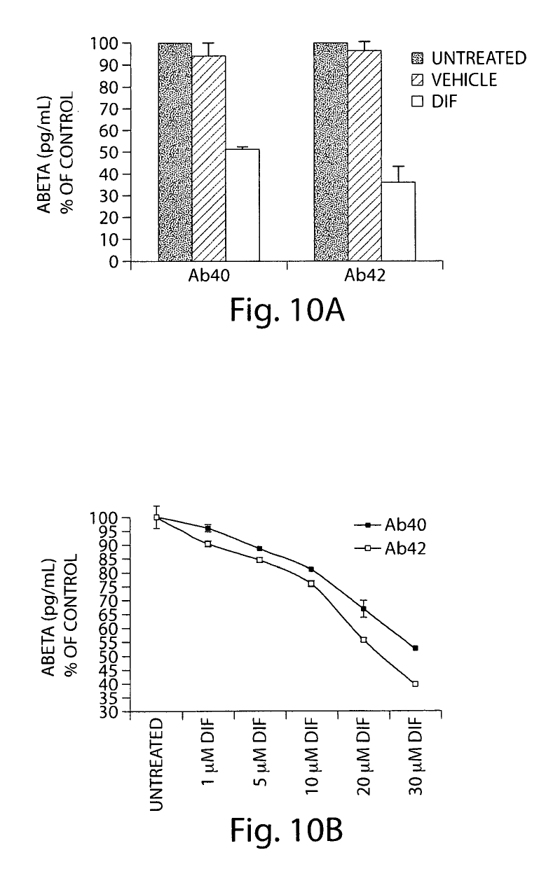

The sandwich ELISA analysis was employed to examine dose-dependency of the DIF-1 effect on the levels of secreted Aβ40 and Aβ42. First, CHO-7W cells were enumerated and the same number were deposited into 6-well dishes and allowed to recover overnight. The next morning, the media was removed and fresh media (2 mL) was added to each well. Cells were treated overnight in triplicate with 30 μM of DIF-1. Media was collected from DIF-treated, non-treated and vehicle treated cells and used to calculate the amount of Aβ40 and Aβ42 by sandwich ELISA. Results are summarized in FIG. 10A. No difference was detected between non-treated (black bars) and vehicle treated (grey bars) cells. Aβ40 was reduced on average by about 48.5±0.91%; where as a greater reduction in Aβ42 was observed (63.9±7.15%).

Alternatively, CHO-CAB cells were treated with increasing concentrations of DIF-1 (1-30 μM) and the levels of secreted Aβ40 and Aβ42 were measured as performed for the CHO-7W cells. The results shown in FIG. 10B represent 3 separate experiments analyzed in triplicate. All media sample were normalized to the detectable level of Aβ in naïve CHO cells, the total protein and cell number post-treatment.

Taken together, analysis of Aβ levels using the BioSource Aβ ELISA kit, further verified that DIF-1 dose-dependently reduced the secreted levels of Aβ40 (44.2±3.4%) and Aβ42 (66.9±2.6%) in conditioned media collected from either CHO-7W or CHO-CAB cells (FIG. 10). Thus it appears that the mechanism by which DIF-1 affects APP metabolism results in a significant differential reduction of Aβ42 levels when compared to that of Aβ40.

### Phosphorylation of APP-CTFs is Inhibited by DIF-1

A number of reports have identified elevated levels of T668-phosphorylated APP CTFs in the hippocampus in AD brains compared to age-matched control brains. This type of posttranslational modification of APP has been suggested to participate in the neuronal dysfunction that accompanies AD (Lee et al., 2003). In keeping with this, phosphorylation of T668 might lead to increased γ-secretase cleavage of APP CTFs (Vingtdeux et al., 2005). This suggests that neuronal signaling networks that lead to an increase in T668 phosphorylation may contribute to the overproduction of Ap species. An understanding of the biological importance of APP phosphorylation and its regulation may lead to potentially novel AD therapies. The effect of DIF-1 imparts a profound decline in the production of APP CTFs, and a significant differential reduction in Aβ42 levels in vitro.

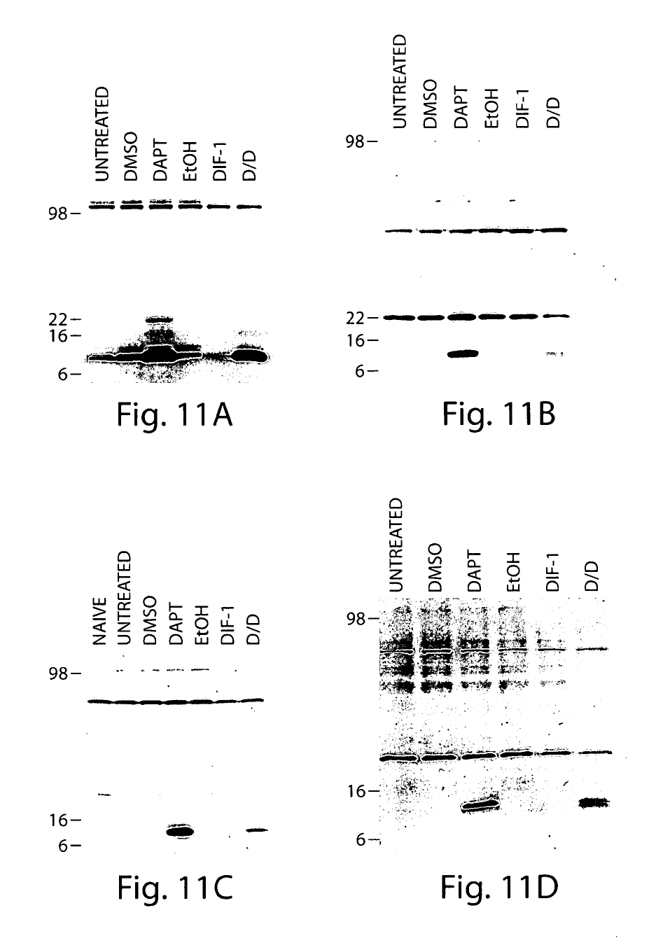

Next, whether or not DIF-1 treatment affected the degree of T668 phosphorylation was examined by western blot (FIG. 11). Briefly, effects of DIF-1 on APP T668 phosphorylation were compared between vehicle-treated control and 250 nM of DAPT identically in each cell line. Western analysis of 15 μg of total protein was probed overnight at 4° C. with anti-APP Cterminal antibody (C66-676-695) (1:5000) and then detected on film using ECL and HRP-conjugated secondary antibodies (1:5000). CHO-CAB cells when treated with DAPT also produce a CTF band of approximately 22 kDa that is also reduced by DIF-1 treatments. This band has also been detected in other CHO lines where APP expression is high (˜3 fold) and by different C-terminal APP antibodies (Ab8717-676-695; except 6E10 which recognizes the N-terminal residues 1-17 of the Aβ sequence. For FIG. 11A, cells were treated with DAPT, and DAPT+DIF then were probed with anti-APP antibody (C66). For FIG. 11B, CHO-7W cells were treated with DAPT, and DAPT+DIF, and then probed with polyclonal anti-P668 APP antibody (1:1000) overnight at 4° C. in 5% BSA. After washing, cells were probed with a rabbit HRP-conjugated secondary antibody (1:5000) in BSA for 1 hour at room temperature and signals were detected on film using ECL. FIG. 11C represents CHO-CAB cells and FIG. 11D represents H4 cells expressing APP^{SW}. In each cell line, DIF-1 reduced the level of detectable T668 on average 86.63±3.16% compared to DAPT alone.

The basal level of APP T668 phosphorylation that occurs in CHO-7W and CHO-CAB cells is below the detection limits of anti-pAPP-T668 antibodies as assessed by western blotting. If T668 phosphorylated APP is a preferred substrate for γ-secretase cleavage as suggested (Vingtdeux et al., 2005), then one would expect that the levels of phosphorylated CTFs would likely be difficult to detect by western blotting, and decreased phosphorylation may accompany a decrease in Aβ production. To circumvent this limitation, it may be reasoned that DAPT inhibition of γ-secretase should increase the total amount of detectable phosphorylated T668 APP CTFs. As predicted, treatment with DAPT greatly increased the amount of detectable phosphorylated APP at residue T668 by western blot using polyclonal anti-pAPP-T668 antibodies (FIG. 11). However, co-treatment of CHO-7W and CHO-CAB cells with DAPT and DIF-1 (30 μM) resulted in a large reduction in the amount of T668 phosphorylated APP CTFs (FIG. 11). This effect of DIF-1 was observed in all cells tested including H4 neuroglioma cells that stably express APP⁷⁵¹ or the Swedish mutant form of APP^{SW} (FIG. 11). Thus, DIF-1 not only reduces the production of both APP CTFs, it consistently reduced the detectable levels of APP T668 phosphorylation on average by 86%±5.6 when compared to DAPT treatment alone. These finding are in agreement with previous reports and suggest APP phosphorylation represents a physiological mechanism that regulates APP metabolism. It also supports the idea that the effect of DIF-1 on CTF levels and Aβ production is not likely to be a result of DIF-1 mediated γ-secretase dysfunction.

### The Effects of Specific Kinase Inhibitors on DIF-Induced Reduction of APP-CTFs

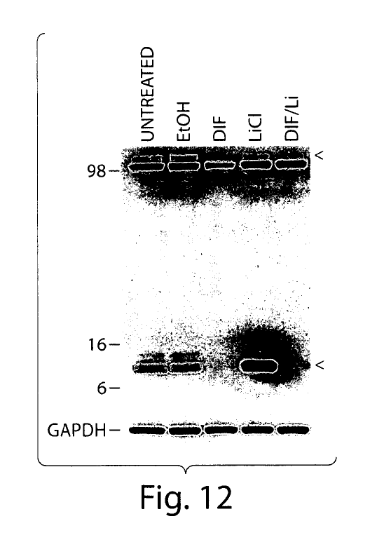

A number of protein kinases have been implicated in the phosphorylation state of APP at residue T668 including GSK3β, cdk5 and JNK3 (Aplin et al., 1996; Iijima et al., 2000; Standen et al., 2001; Kimberly et al., 2005) yet the physiological role associated with T668 phosphorylation is still very much a matter of debate. Because DIF-1 drastically reduces the level of T668 phosphorylated APP (86.63±3.16%), we assessed a number of specific kinase inhibitors for their ability to attenuate or potentiate the effect of DIF-1 on APP metabolism. We hoped that this might identify a potential mechanism for the observed effects of DIF-1. Since it has been reported that the cytostatic effect of DIF-1 might be through the activation of GSK3β, a number of specific GSK3β inhibitors were assessed to modulate the effect of DIF-1 on APP metabolism. CHO-7W cells were treated overnight with non-toxic concentrations of lithium (5-10 mM) and the effect on APP processing was analyzed by western blot. Cells were pretreated for four hours with LiCl before the addition of 30 μM DIF-1. 15 μg of total protein was loaded and probed overnight at 4° C. with anti-aPP antibody C66 (1:5000), washed and probed with polyclonal HRP-conjugated secondary antibodies (1:5000) for 1 hour at room temperature in 5% non-fat milk then detected on film using ECL. Lithium chloride (LiCl) is known for its potent ability to inhibit GSK3β activity and has been reported to reduce Aβ production in cultured neurons and different cellular models (Phiel et al., 2003; Su et al., 2004). However, in these studies the cells were treated with levels of LiCl that in our system showed cytotoxicity and as such could lead to the reported decrease in Aβ production. Similar findings in support of our results have also been reported where therapeutic levels of LiCl led to increases in βCTF levels in CHO cells expressing APP⁶⁹⁵ (Feyt et al., 2005). When CHO-7W or CHO-CAB cells were treated with 1-10 mM of LiCl it caused an increase both αCTF and βCTF levels compared to control cells treated with NaCl (FIG. 12). This effect of lithium treatment is indicated in FIG. 12 (arrowhead). As shown in FIG. 12, Lithium treatment did not significantly prevent the effect of DIF-1 on APP maturation (arrowhead). Pretreatment of cells with LiCl and DIF-1 caused a minor increase in αCTF levels, but could not rescue the effect of DIF-1 on APP maturation or βCTF levels (FIG. 12; arrowhead). We then examined the effect of two different GSK3β inhibitors, kenpaullone and 1-azakenpaullone, which have been shown to be much more specific at inhibiting GSK than LiCl (Bain et al., 2003; Kunick et al., 2004). The combined use of kenpaullone, 1-azakenpaullone and LiCl has been shown to be far more useful in identifying both substrates and the physiological roles of GSK3α/β (Bain et al., 2003). Kenpaullone and 1-azakenpaullone treatments, unlike LiCl, had no effect on the level of APP CTFs. Furthermore, pretreatment with both GSK3β inhibitors and DIF-1 neither blocked nor potentiated the effect of DIF-1 on APP CTF production. This suggests that the increase in APP CTF levels in response to LiCl treatment may be due to effects independent of GSK3α/β. In order to determine if the effect of DIF-1 involved cyclin-dependent kinase 5, we treated each CHO cell line with the potent cdk5 inhibitor roscovitine. The combined use of roscovitine and kenpaullone is useful for identifying substrates and physiological roles of cyclin-dependent protein kinases rather than using each inhibitor alone (Bain et al., 2003). Roscovitine alone had no effect on APP metabolism. Pretreatment of cells with roscovitine and DIF-1 neither blocked nor potentiated the effect of DIF-1 on APP CTF production. Since JNK3 has also been suggested as another kinase likely responsible for the phosphorylation of APP at T668 (Kimberly et al., 2005) we assessed the effect of the specific JNK inhibitor SP600125 on APP metabolism. Cells treated with SP600125 had no effect on the level of APP processing compared to control cells. However, pretreatment with 10 μM SP600125 and 30 μM DIF-1 did not block or potentiate the effect of DIF-1 on APP CTF production, but it did lead to a rescue of the level of mature APP as detected by western blot. Cells were then treated with 10 μM SP600125 in the presence of increasing concentrations of DIF-1 (5-30 μM). Interestingly the presence of 10 μM SP600125 blocked the effect of APP maturation for each concentration of DIF-1, but had no effect on the decrease of either αCTF or βCTF levels.

### Mutation of APP T668A and T668E Abolished the Effect of DIF-1 on APP Maturation and Metabolism

All of the kinase inhibitors used in this study, even after four hour pretreatments, could not disrupt the ability of DIF-1 to reduce the level of APP CTFs and ultimately inhibit the production of Aβ. A number of possibilities might exist that may explain the observed effect of DIF-1 on APP metabolism. DIF-1 might act as an inhibitor of an unknown kinase of which APP is a substrate; the effects of DIF-1 on the phosphorylation state of T668 might be a due to an increase in the activity of a G0/G1 cell cycle-dependent APP phosphatase, or, DIF-1 perturbs APP processing by a yet to be identified mechanism that is dependent on the phase of the cell or the state of T668 phosphorylation. In order to determine the importance of the T668 residue with respect to the mechanism of DIF-1 on APP metabolism, we made two mutant APP⁷⁵¹ molecules that cannot be phosphorylated at T668 (T668A and T668E). CHO cells that stably express either the T668A or T668E mutation were created and hypothesized that DIF-1 should have a diminished effect on the processing of APPT668E since the glutamic acid residue should function as a mimic of permanent APP phosphorylation. The effect of 30 μM of DIF-1 on APP metabolism was assessed for each of the mutant APP T668 constructs by western blot. Surprisingly, both mutant APP molecules (T668A or T668E) completely abolished the effect of DIF-1 on APP maturation and the reduction of both αCTF and βCTF levels. This result was entirely unsuspected since the T668E mutation should represent a cell cycle-independent phosphorylated state of APP. However, it suggests that the T668 residue is more important with respect to the mechanism of DIF-1 on APP metabolism. The use of DIF-1 combined with the replacement of T668 with a glutamic acid residue may have revealed a constraint or preference acting on a particular physicochemical property at the amino acid level. Such properties may confer a previously unidentified structural integrity of the APP C-terminal domains in its native phospho-state. A growing number of adapter proteins such as such as mDab1, Fe65, JIP1 and X11α have been shown to interact phospho-independently with the C-terminal domain typically requiring the 681-GYENPTY-687 motif [SEQ ID NO:12] (Borg et al., 1998; Fiore et al., 1995; Scheinfeld et al., 2002). Yet some debate remains as to the effect of the phosphorylation state of APP at T668 and its' ability to modulate the binding of adapter proteins. In keeping with, our analysis further suggests that the S/TP region encompassing T668 is a class IV WW phosphorylation-dependent interaction motif potentially recognized by class IV WW domain proteins. Theoretical mutation of the threonine to any other residue (except serine) upstream of the proline residue no longer predicts this potential motif. Sequence analysis shows that both APP and APLP2 contain the S/TP motif and, experimentally we have shown that DIF-1 effects the metabolic processing of each protein. However, APLP1 does not contain an S/TP motif due to the lack of the proline residue, and we have shown that DIF-1 has no effect on APLP1 metabolism. Interestingly, it was reported that the peptidyl-prolyl cis/trans isomerase Pin1 binds T668 phosphorylated APP (Pastorino et al., 2006). Pin1 differs from other PPIases because it only isomerizes the bond between pSer/Thr-Pro motifs (Lu et al., 2002; Schutkowski et al. 1998). We hypothesize that the effect of DIF-1 on APP/APLP2 metabolic processing occurs through a novel mechanism that requires the S/TP motif, and that this mechanism has only been revealed through the use of DIF-1 as a tool to understand APP metabolism.

### DIF-1 Reverses Proteasome-Mediated Accumulation of APP CTF Levels

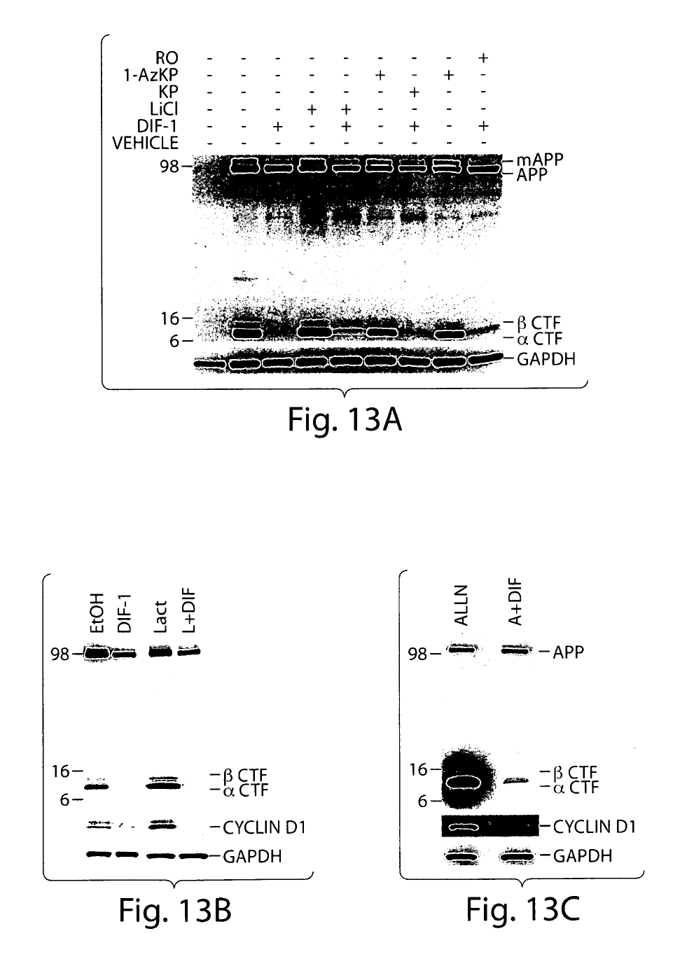

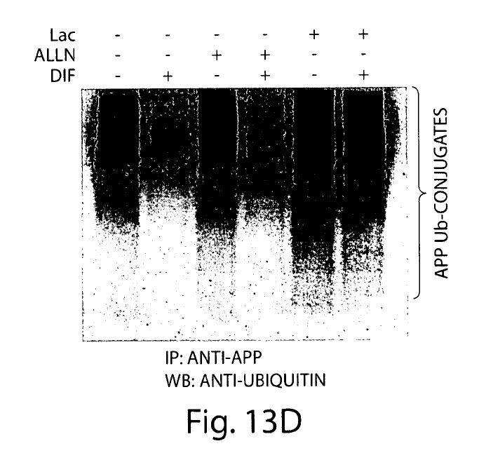

The effect of cell cycle/GSK kinase inhibitors and proteasome inhibition on the DIF-1 dependent reduction in APP processing was examined. As shown in FIG. 13A, three specific GSK kinase inhibitors (LiCl, kenpaullone and 1-azakenpaullone) were assessed in the presence of DIF-1. CHO-CAB cells were treated with lithium chloride (10mM). Kenpaullone and 1-azakenpaullone (10 μM) had no effect on APP processing, and neither inhibitor could restore APP CTF levels in the presence of DIF-1. The Cdk5 inhibitor roscovitine (10 μM) was used as a control to ensure APP processing by LiCl, kenpaullone or 1-azakenpaullone was not a result of non-specific inhibition of Cdk5. FIG. 13B shows that the effect of proteasome inhibitors on DIF-1 processing of APP. Treatment with lactacystin (10 μM) induced an increase in the level of αCTF and βCTF compared to vehicle-treated cells. However, DIF-1 (30 μM) greatly reduced the effect of lactacystin. We then assessed the effect of a second proteasome inhibitor, ALLN, on APP processing (FIG. 13C). ALLN (10 μM) also induced an increase in CTF levels compared to vehicle treated cells. Co-treatment with DIF-1 reduced the effect of ALLN on APP processing. To ensure that the results observed were due to proteasome inhibition, we assessed the effects of lactacystin and ALLN on cyclin D1 levels. Cyclin D1 is targeted for degradation via ubiquitin-mediated 26S proteasome activity. Both lactacystin and ALLN treatments increased the levels of cyclin D1. However, co-treatment with DIF-1 inhibited the accumulation of cyclin D1 for each proteasome inhibitor used. As shown in FIG. 13D, DIF-1 affects the cellular level of APP ubiquitin-conjugates. CHO-CAB cell protein extracts (1 mg) were immunoprecipitated using APP antibodies. The immunoprecipitated complexes were analyzed by western blotting using anti-ubiquitin antibodies. Treatment with proteasome inhibitors lactacystin and ALLN increased the level of APP ubiquitin conjugates. DIF-1 treatment results in decreased levels of APP ubiquitin-conjugates compared to vehicle treated cells. Co-treatment with DIF-1 and ALLN prevented the accumulation APP ubiqutin-conjugates. Molecular Weight markers (kDa) are shown on the left. GAPDH levels are shown below each blot. The data represents that of three separate experiments.

A few kinases including GSK3β and cdk5 have been shown to phosphorylate APP at T668 (Aplin et al., 1996; Iijima et al., 2000). Since DIF-1 reduces pAPPT668 and reports suggest that DIF-1 activates GSK3β (Yasmin et al., 2005), we examined the effects of three structurally distinct GSK inhibitors (LiCl, kenpaullone and 1-azakenpaullone) on DIF-1 processing of APP (shown in FIG. 13A). LiCl (10 mM) treatments alone increased both αCTF and βCTF levels compared to control cells, whereas LiCl and DIF-1 together resulted in only a slight increase in αCTF levels (FIG. 13A). In contrast, neither of the more potent and specific cell cycle/GSK inhibitors kenpaullone (10 μM) and 1-azakenpaullone (10 μM) (Crews et al., 2003) had an effect on APP processing compared to LiCl (FIG. 13A). Furthermore, kenpaullone or 1-azakenpaullone neither attenuated nor potentiated the effect of DIF-1 on APP processing (FIG. 13A). Thus; it appears that the effect of DIF-1 on APP processing is likely not due to increased GSK3α/β activity in CHO cells. In fact, the effect of DIF-1 was not affected by any kinase inhibitors of which the kinase has been implicated in T668 phosphorylation. The effects of DIF-1 on APP processing compared with those of kenpaullone, 1-azakenpaullone and roscovitine which also affect cell cycle progression (G0/G1, G2 and M phase) (Crews et al., 2003), suggests the effect of DIF-1 might be due to G0/G1-associated metabolic pathways.

The group IV WW-protein interaction motif (Ser/Thr-Pro) has been shown to serve as a potential signaling module in the regulation of protein degradation via the proteasome (Lu et al., 1999; Verdecia et al., 2000) and the finding that T668A and T668E mutations abolish the effect of DIF-1, suggest that this small domain is critical for the effect of DIF-1 on APP processing. To determine if the proteasome is involved in this process, CHO-CAB cells were pre-treated with two structurally different proteasome inhibitors. Lactacystin (10 μM) and Acetyl-L-Leucyl-L-Leucyl-L-Norleucinal (ALLN) (10 μM) were assessed for their effect on APP processing alone or combined with DIF-1 by western blotting (FIGS. 13B & 13C). As a positive control for proteasomal degradation, we also monitored protein levels of cyclin D1, which appears to be solely degraded by the proteasome (Alao et al., 2007) (FIGS. 13B & 13C). As shown, cyclin D1 levels were increased when cells were treated with either lactacystin or ALLN. Proteasomal inhibition by lactacystin increased APP CTF levels compared to control cells (FIG. 13B). Interestingly, co-treatment with DIF-1 greatly diminished the effect of lactacystin on APP CTF accumulation (FIG. 13B). However, even when cells are pre-treated with lactacystin, the addition of DIF-1 results in the reduction of APP CTF levels (FIG. 13B). We next assessed the effect of ALLN on the accumulation of APP CTFs. FIG. 13C shows that low concentrations of ALLN (10 μM) led to an accumulation of APP CTF levels (FIG. 13C). Cells were pre-treated with ALLN prior to the addition of DIF-1. Consistent with our observation using lactacystin, the addition of DIF-1 resulted in a reduction of APP CTFs (FIG. 13C). Taken together, it appears that DIF-1 may increase proteasomal activity, or might increase the affinity of APP or APLP2 for an unknown, proteasome-associated group IV WW domain-containing protein via the (Thr668-Pro) motif.

Degradation by the proteasome is thought to be triggered by the polyubiquitination of a target protein. To determine whether the proteasome-mediated degradation of APP is accompanied by increased levels of poly-ubiquitinated APP, we investigated the ubiquitination status of APP in extracts of CHO-CAB cells treated in the absence or presence of DIF-1 (30 μM), lactacystin (10 μM), ALLN (10 μM), DIF-1+lactacystin and DIF-1+ALLN. Upon subsequent immunoprecipitation with an anti-APP antibody (8717), followed by Western blot using monoclonal ubiquitin antibody, polyubiquitinated APP is clearly visible in CHO-CAB cell extracts (FIG. 13D). Upon treatment with DIF-1, the amount of APP ubiquitin-conjugates is decreased below the basal level seen in vehicle treated cells. As shown, treatment of cells overnight with the proteasome inhibitors lactacystin and ALLN resulted in an increase in the level of APP ubiquitin-conjugates (FIG. 13D). In comparison, DIF-1 prevented the accumulation APP ubiquitin-conjugates when used together with ALLN. However, the level of APP ubiquitin conjugates was only slightly reduced in cells treated with both lactacystin and DIF-1. Thus the proteasome-dependent degradation of APP in CHO cells is accompanied by ubiquitin ligase activity towards APP, and it is consistent with the notion that DIF-1 enhances the degradation of polyubiquitinated APP.

### DIF-1 Alters the Cellular Distribution of APP

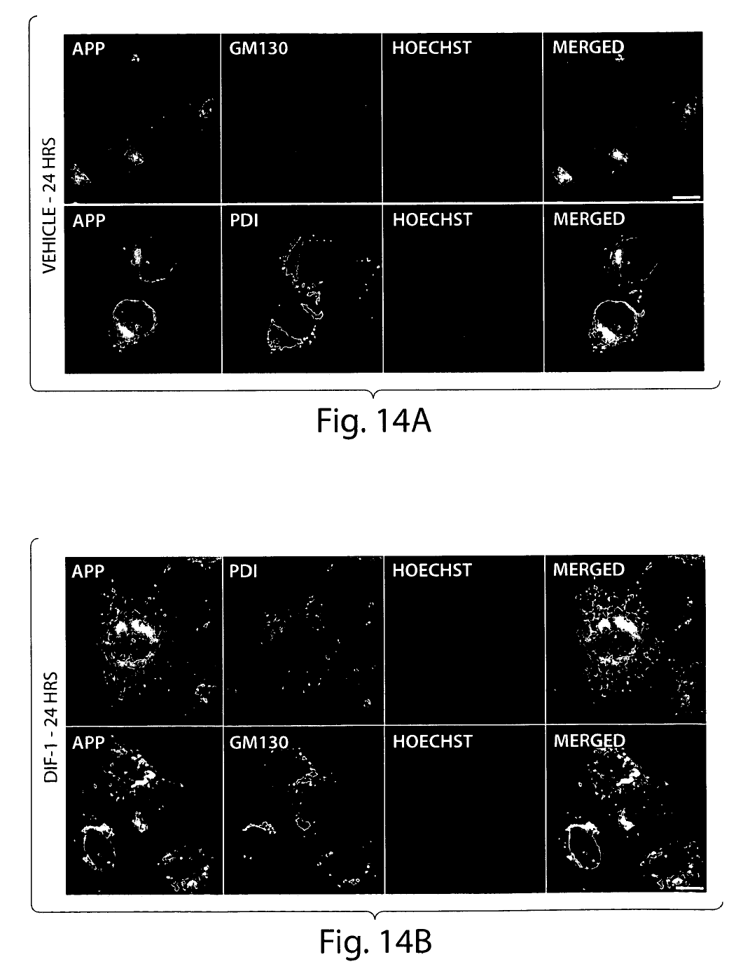

Next, the effect of DIF-1 on APP trafficking and subcellular distribution was assessed. Cells were fixed with 4% paraformaldehyde/PBS, permeabilized with 0.1% Triton X-100/PBS and blocked with 5% goat serum/PBS. Confocal microscopy of CHO-CAB cells stained with APP antibodies together with the ER marker PDI or golgi marker GM130 confirmed that APP primarily co-localized with GM130 and PDI (FIG. 14A). Treatment with DIF-1 did not affect the cellular distribution of GM130, but did cause a disruption in the structure of the ER (FIG. 14B). ER structure upon DIF-1 treatment became highly vacuolated but to a varying degree within populations of cells. Further to this, APP was found to be enriched around the nuclear periphery (FIG. 14B). Taken together, the data suggests that DIF-1 impedes APP trafficking to the Golgi and potentially the secretory system resulting in reductions of mAPP and Aβ.

### DIF-1 Increases the Co-Localization of Cellular APP and Ubiquitin

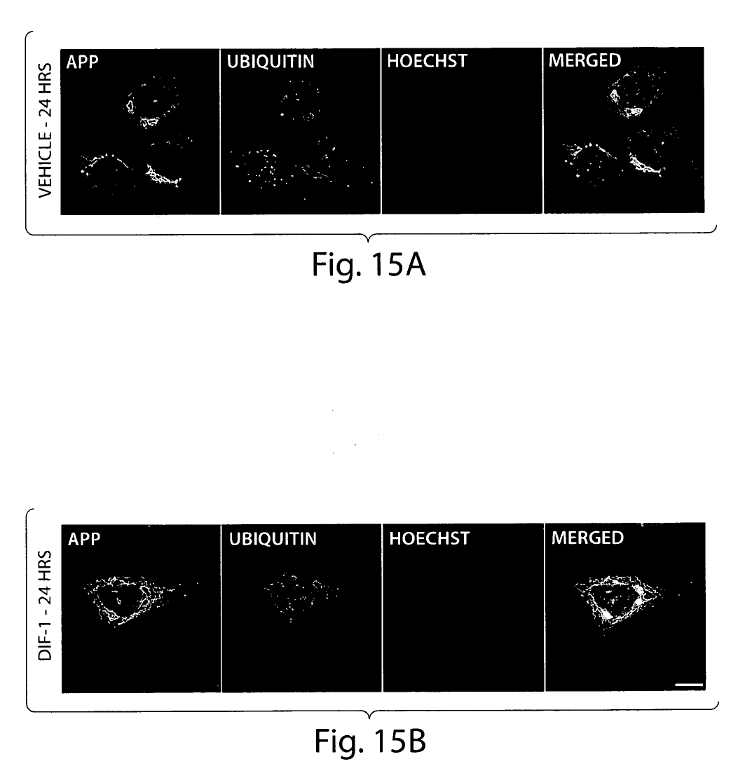

The finding that DIF-1 prevents the accumulation of APP ubiquitin-conjugates suggested that this small-molecule enhances an ubiquitin-dependent ALLN-sensitive protein degradation pathway. Therefore, the subcellular localization of ubiquitin and APP in CHO-CAB cells in response to DIF-1 was examined. Confocal microscopy of CHO-CAB cells depict regions of the Golgi that partially co-stain with APP and ubiquitin (FIG. 15A). Ubiquitin staining is also apparent as punctuate spots in the nucleus (FIG. 15A). Treatment with DIF-1 resulted in staining of APP and ubiquitin around the nuclear periphery (FIG. 15B). This suggests that DIF-1 might potentiate the co-localization of APP with ubiquitin or ubiquitin conjugating enzymes to enhance proteasomal degradation of APP.

### Discussion

### The Importance of Cell-Cycle Dependent Modulators of APP Metabolism and Aβ Production

Outside of neurons, cycling cells respond to and receive mitotic signaling that carefully regulates how and when checkpoint proteins are either turned on or off to either initiate or complete DNA replication (S phase) (Murray, 1992). The existence of sites of chromosomal replication, S phase hallmarks and the aberrant expression of cyclin D1 seen in the hippocampal neurons from postmortem tissue of AD brain (Busser et al., 1998) should intensify the experimental approach at elucidating the chain of events that just might be ushering in the progressive neurodegeneration that occurs in AD. A highly compelling, but recent discovery that favors theories on aberrant cell cycle progression in AD is the finding that some chromosomes are either partially or fully replicated in a significant quantity of AD neurons (Yang et al., 2001). In contrast neurons of non-demented, age-matched individuals typically have a normal diploid DNA content. Further to this, the Cdk4 kinase and Cyclins Dl are all upregulated (Tsujioka et al., 1999; McShea et al., 1997; Busser et al., 1998). In AD the upregulation of cyclins are particularly found to be associated with NFT-containing neurons (McShea, 1997). Of importance and interest is that other S phase associated proteins including Ki67, a proliferation marker and known modulator of the speed in which a tumor grows (Nagy et al., 1997; Pich et al., 2004) and PCNA (Busser et al., 1998) have also been confirmed to reside AD afflicted neurons. Although this number of identified Gl/S checkpoint proteins definitely doesn't represent the whole repertoire of known molecules believed to organize and coordinate the proliferative response in dividing cells, their discovery and that of duplicated genomic DNA in AD neurons provides hints that those neurons encompass enough of the prerequisites to attempt cell cycle entry and ultimately DNA replication. A long-lasting understanding of neurons is that they do not replicate their DNA, and they cannot proceed through mitosis. Any attempt, in vitro or in vivo ultimately initiates neuronal degeneration followed by programmed cell death. Even the minor amounts of replicated DNA detected in AD brain are enough evidence to ask why it is there, and what are the circumstances to blame for activating cell cycle entry in postmitotic neurons.

### The Role of Posttranslational Modifications in APP Processing, Trafficking and Secretion

APP is a type I membrane-spanning protein whose secretion is regulated by a variety of factors including growth factors, neurotransmitters, phorbol esters, extracellular matrix molecules, and stress (Schubert et al., 1989; Mills and Reiner, 1999; De Strooper and Annaert, 2000). The mechanisms involved in the regulation of APP metabolism include alternations in APP phosphorylation (Caporaso et al., 1992), the modification of protein glycosylation (Galbete et al., 2000), alternative gene splicing (Shepherd et al., 2000) transcription (Ciallella et al. 1994), and also changes in protein degradation (Checler et al., 2000). Although most evidence points to secretion of Aβ as an underlying cause of AD, the underlying cell biology of APP metabolism is still not fully understood. It has been shown that APP is phosphorylated at T668 by a number of kinases in vitro, and that this event likely occurs in a cell cycle-dependent manner in vivo (Suzuki et al., 1994). The maximal level of APP phosphorylation has been reported to happen in the G2/M phase of the cell cycle (Suzuki et al., 1997). This report has implied the imaginable scenario that phosphorylation-dependent events which occur during cell cycle progression influence the metabolism of APP. In support of this, the highly related amyloid precursor like protein APLP2 is also phosphorylated at a complementary site and is also maximal during M phase (Suzuki et al., 1997). However, this seems counterintuitive to the etiology of AD. Neurons are not supposed to cycle and they most certainly cannot proceed through mitosis. Reports have suggested that APP is not only subjected to phosphorylation, but that AD brains contain higher amounts of T668 phosphorylated (Lee et al., 2003). Furthermore, it has been suggested that phosphorylated APP at T668 not only makes it a better substrate for BACE1 cleavage, it may also serve to facilitate CTF cleavage by g-secretase in primary cultures of embryonic rat cortical neurons and the human neuroblastoma cell line SYSY (Lee et al., 2003; Vingtdeux et al., 2005). Indirectly, this evidence of increase APP phosphorylation and its reported effect on increase Aβ production supports the hypothesis that subpopulations of neurons in AD brain are experiencing uncontrolled cell cycle reentry. Our findings presented above support the notion that a complex etiology, environmental insults and increased risk factors associated with aging may influence aberrant cell cycle regulation in both sporadic and early onset AD ultimately causing slow progressive neuronal degeneration. A point of further interest is that outside of neurons, all non-neuronal cells cycle at intrinsic rates. Controlled cell division allows for growth, development, and healing. This physiological process is highly protective against the formation of tumors. Aberrant cycling or acquired insults to cell cycle checkpoints allow for the induction of apoptosis which effectively removes the unhealthy cell. Only after accumulations of many mutations can cycling cells become transformed. This too has both a genetic and age-dependent sporadic nature with the most common result being the formation of tumors. However, the intrinsic neuronal post-mitotic block, when forced through the G1/S checkpoint has no option but to die leaving a hole where a memory once lived.

### The Mechanism of DIF-1 on APP Processing and Aβ Production

Understanding AD and neurodegeneration within the framework of cellular differentiation and cell division control is not a new idea. Accumulating evidence generated from models of AD, suggest that mitogenic pathways are specifically activated in a select population of neurons in defined regions of the brain early on in the development of AD (Gartner et al., 1995). Yet the biological processes are still unclear as to how cellular differentiation is regulated in normal or diseased neurons, and to what extent the expression of cell cycle proteins contribute to AD pathology. It would appear that a more complete understanding of AD might come from an unlikely resource, that being the biology of cancer research. A huge benefit in making this connection is that it creates the opportunity to examine a whole host of anti-tumor candidate drugs in the treatment of AD. A potential long-term benefit would be a provisional approach for treating two of the most costly health problems facing society today.

Structural Motifs within Proteins that Influence their Degradation and Involvement in Neurodegenerative Disease

Our finding that the DIF-1 induced degradation of cyclin D1 is concomitant with a decrease in APP processing and reduced levels of Aβ42 may provide new avenues of research into the proteasomal-dependent degradation of APP. However, the effect of DIF-1 on APP maturation and CTF levels may involve increased proteasomal activation or an increase in the association of a specific E3-ubiquitin ligase that targets APP for degradation effectively reducing its' trafficking into the secretory system. Indeed, data presented herein suggest DIF-1 works in part by enhancing ubiquitin-dependent ALLN-sensitive protein degradation pathways. Since DIF-1 did not affect the level processing of the highly homologous APLP1 suggests that the effect of DIF-1 is likely not just a general increase in proteasomal activity. Furthermore, the mechanism by which DIF-1 accelerates cyclin D1 degradation by the proteasome prior to entry into S phase is still poorly understood since the levels of cyclin D1 could not be restored by pretreating cells with three different inhibitors of GSK3β. Our findings are different from previous reports that suggest DIF-1 activates GSK3β, which increases the phosphorylation of cyclin D1 at T286 thus targeting it for degradation (Yasmin et al., 2005). This may be due to the inherent differences in cell types used in each study, but is likely due to our overnight treatments compared to three hours. Notebly, phosphorylation of APP has also been implicated in Aβ generation and transport to neurite endings (Lee et al., 2003; Vingtdeux et al., 2005; Iijima et al., 2000). Moreover, Phosphorylation of APP has also been implicated in Aβ generation and transport to neurite endings (Lee et al., 2003; Vingtdeux et al., 2005; Iijima et al., 2000).

Because APP binds to the molecular chaperones Bip/G-RP78 and HSC73 and misfolded proteins bound to Bip/GRP78 are degraded, it has been suggested that APP can be retained in the ER as a nascent polypeptide and degraded (Yang et al., 1998; Kouchi et al., 1999) which is consistent with data suggesting that the proteasome is involved in APP processing (Hare, 2001). The ubiquitin-proteasome system is abnormal in AD brains and implicated in the turnover of proteins directly responsible for metabolizing APP. A targeted siRNA screen of ubiquitin ligases involved in APP metabolism identified a peptidylprolyl isomerase (PPIL), which reduces Aβ production by suppressing beta-site cleavage (Espeseth et al., 2006). However, specific APP ubiquitin ligases responsible for proteasomal degradation have yet to be discovered.

The abbreviations used herein are as follows: AD, Alzheimer's disease; PHF, paired helical filament; GSK3β/α, glycogen synthase kinase 3-beta/alpha; Aβ, amyloid peptide; APP, amyloid precursor protein; APLP1, amyloid precursor-like proteins 1; APLP2, amyloid precursor-like proteins 2; saAPP, soluble α-amyloid precursor protein; sβAPP, soluble β-amyloid precursor protein; BACE1, β-site APP-cleaving enzyme 1; αCTF, C-terminal fragment of APP produced by a-cleavage; βCTF, C-terminal fragment of APP produced by β-cleavage; γ-secretase, gamma-secretase composed of the proteins presenilin 1 or 2, nicastrin, anterior pharynx-defective 1a and presenilin enhancer 2; LiCl, lithium chloride; DAPT, N4-[N-(3,5-difluorophenacetyl-1-alanyl)]-S-phenylglycine tert-butyl ester; PS1/PS2, presenilin 1/presenilin 2; PBS, phosphate-buffered saline; PCNA, Proliferating Cell Nuclear Antigen; MCI, mild cognitive impairment; DIF-1, Differentiation-inducing factor-1 (1-[3,5-dichloro-2,6-dihydroxy-4-methoxyphenyl]-1 hexanone); CHO, Chinese Hamster Ovary; MEF, mouse embryonic fibroblast; NICD, notch intracellular domain; ELISA, enzyme-linked immunosorbent assay; T, threonine; A, alanine; E, glutamic acid; cdk5, cyclin-dependent kinase 5; JNK, c-Jun NH2-terminal kinase; Pinl, peptidyl prolyl cis/trans isomerase; S/TP, serine/threonine proline; NFT, neurofibrillary tangles; FAD, Familial Alzheimer's disease; EOAD, early-onset Alzheimer's disease; C83, C-terminal 83 amino acids produced by a-cleavage of APP; C99, C-terminal 99 amino acids produced by b-cleavage of APP; CDK4, cyclin-dependent kinase 4; ALS, Amyotrophic lateral sclerosis; ADAM10, A disintegrin and metalloproteinase domain 10; pstO, pre-stalk cell type O; FD, furanodictine; siRNA, Small interfering ribonucleic acid; Tcf-Lef transcription factors, human T-cell factor-mouse lymphoid enhancer factor; SH-SY5Y, 3rd generation human neuroblastoma from SH SY5 which is from SH-SY derived from SK-N-SH; kenpaullone, 9-bromo-7,12-dihydro-indolo[3,2d][1]benzazepin-6(5H)-one (IC50 0.023 uM GSK/0.85 CDK5); 1-azakenpaullone, 8-bromo 6,11-dihydrothieno[30,20:2,3]azepino[4,5-b]indol-5(4H)-one (IC50 0.018 uM GSK/4.2 CDK5; roscovitine, 2-(R)-[[9-(1-Methylethyl)-6-[(phenylmethyl)amino]-9H-purin-2-yl]amino]-1 butanol; SP600125, 1,9-Pyrazoloanthrone; LAMP2, Lysosome-associated membrane protein 2; GAPDH, glyceraldehyde-3-phosphate dehydrogenase; PDI, Protein Disulphide Isomerase; GM 130, cis-Golgi matrix protein in Golgi bodies; MG-132, (N-carbobenzoxyl-Leu-Leu-leucinal); ALLN, Acetyl-L-Leucyl-L-Leucyl-L-Norleucinal; EDTA, Ethylene diamine tetracetic acid; lactacystin, 3S-hydroxy-2R-(1-hydroxy-2-methylpropyl)-4R-methyl-5-oxo-2 pyrrolidine-carboxylate-N-acetyl-L-cysteine; PVDF, Polyvinylidene Difluoride; BCA, bicinchoninic acid; ECL, enhanced chemiluminescence; DMSO, dimethyl sulfoxide; EtOH, ethanol; PCR, polymerase chain reaction; LB, Luria-Bertani broth; RIPA, Radio-Immunoprecipitation Assay; SDS-PAGE, sodium dodecyl sulfate polyacrylamide gel electrophoresis; TTBS, Tween 20 0.05% (v/v) Tris-buffered saline pH 7.5; NaCl, sodium chloride.

### REFERENCES

Nagy, Z., Esiri, M. M., and Smith, A. D. (1997) Acta Neuropathol (Berl) 93: 294-300.

Pich A, Chiusa L, Navone R. (2004) Prognostic relevance of cell proliferation in head and neck tumors. Ann Oncol. 2004 September; 15(9):1319-29.

Suzuki, T., Oishi, M., Marshak, D. R., Czernik, A. J., Nairn, A. C., and Greengard, P. (1994) Embo J 13: 1114-1122.

Yang, Y., Geldmacher, D. S., and Herrup, K. (2001) J Neurosci 21: 2661-2668.

Lee M S, Kao S C, Lemere C A, Xia W, Tseng H C, Zhou Y, Neve R, Ahlijanian M K, Tsai L H. (2003) APP processing is regulated by cytoplasmic phosphorylation. J Cell Biol. 163(1): 83-95.

Suzuki, T., Ando, K., Isohara, T., Oishi, M., Lim, G. S., Satoh, Y., Wasco, W., Tanzi, R. E., Nairn, A. C., Greengard, P., Gandy, S. E., and Kirino, Y. (1997) Biochemistry 36: 4643-4649.

Lu K. P., Liou Y. C. and Zhou X. Z. (2002) Pinning down proline directed phosphorylation signaling. Trends Cell Biol. 12: 164-172.

McShea, A., Harris, P. L., Webster, K. R., Wahl, A. F., and Smith, M. A. (1997) Am J Path of 150: 1933-1939.

Murray, A. W. (1992) Nature 359: 599-604.

Tsujioka, Y., Takahashi, M., Tsuboi, Y., Yamamoto, T., and Yamada, T. (1999) Dement Geriatr Cogn Disord 10: 192-198.

Schutkowski M., Bernhardt A., Zhou X. Z., Shen M., Reimer U., Rahfeld J. U., Lu K. P. and Fischer G. (1998) Role of phosphorylation in determining the backbone dynamics of the serine/threonine-proline motif and Pinl substrate recognition. Biochemistry 37: 5566-5575.

Borg, J. P., Yang, Y., De Taddeo-Borg, M., Margolis, B., and Turner, R. S. (1998) The X11a Protein Slows Cellular Amyloid Precursor Protein Processing and Reduces A40 and A42 Secretion J. Biol. Chem. 273: 14761-14766.

Fiore F, Zambrano N, Minopoli G, Donini V, Duilio A, Russo T. (1995) The regions of the Fe65 protein homologous to the phosphotyrosine interaction/phosphotyrosine binding domain of Shc bind the intracellular domain of the Alzheimer's amyloid precursor protein. J Biol Chem. 270(52): 30853-6.

Scheinfeld M H, Roncarati R, Vito P, Lopez P A, Abdallah M, D'Adamio L. (2002) Jun NH2-terminal kinase (JNK) interacting protein 1 (JIP1) binds the cytoplasmic domain of the Alzheimer's beta-amyloid precursor protein (APP). J Biol Chem. 277(5): 3767-75.

Kunick, C., Lauenroth, K., Leost, M., Meijer, L., Lemcke, T. (2004) 1-Azakenpaullone is a selective inhibitor synthase kinase-3β. Bioorganic & Medicinal Chemistry Letters 14: 413-416.

Bain J, McLauchlan H, Elliott M, Cohen P. (2003) The specificities of protein kinase inhibitors: an update. Biochem J. 371(Pt 1): 199-204.

Feyt C, Kienlen-Campard P, Leroy K, N'Kuli F, Courtoy P J, Brion J P, Octave J N. (2005) Lithium chloride increases the production of amyloid-beta peptide independently from its inhibition of glycogen synthase kinase 3. J Biol Chem. 280(39): 33220-7.

Phiel, C. J., Wilson, C. A., Lee, V. M., and Klein, P. S. (2003) GSK-3alpha regulates production of Alzheimer's disease amyloid-beta peptides. Nature 423 : 435-439.

Su, Y., Ryder, J., Li, B., Wu, X., Fox, N., Solenberg, P., Brune, K., Paul, S., Zhou, Y., Liu, F., and Ni, B. (2004) Lithium, a common drug for bipolar disorder treatment, regulates amyloid-beta precursor protein processing. Biochemistry 43: 6899-6908.

Aplin, A. E., Gibb, G. M., Jacobsen, J. S., Gallo, J. M., and Anderton, B. H. (1996) J. Neurochem. 67: 699-707.

Iijima, K., Ando, K., Takeda, S., Satoh, Y., Seki, T., Itohara, S., Greengard, P., Kirino, Y., Nairn, A. C., and Suzuki, T. (2000) J. Neurochem. 75: 1085-1091.

Standen, C. L., Brownlees, J., Grierson, A. J., Kesavapany, S., Lau, K. F., McLoughlin, D. M., and Miller, C. C. (2001) J. Neurochem. 76: 316-320.

Kimberly W T, Zheng J B, Town T, Flavell R A, Selkoe D J. (2005) Physiological Regulation of the β-Amyloid Precursor Protein Signaling Domain by c-Jun N-Terminal Kinase JNK3 during Neuronal Differentiation. J. Neuroscience. 25(23): 5533-5543.

Vingtdeux V, Hamdane M, Gompel M, Bégard S, Drobecq H, Ghestem A, Grosjean ME, Kostanjevecki V, Grognet P, Vanmechelen E, Buée L, Delacourte A, Sergeant N. (2005) Phosphorylation of amyloid precursor carboxy-terminal fragments enhances their processing by a gamma-secretase-dependent mechanism. Neurobiol Dis. (2): 625-37.

Lee M S, Kao S C, Lemere C A, Xia W, Tseng H C, Zhou Y, Neve R, Ahlijanian M K, Tsai L H. (2003) APP processing is regulated by cytoplasmic phosphorylation. Cell Biol. 163(1): 83-95.

Gartner, U., Holzer, M., Heumann, R., and Arendt, T. (1995) Induction of p21ras in Alzheimer pathology. Neuroreport. 6(10): 1441-4.

Espeseth A S, Huang Q, Gates A, Xu M, Yu Y, Simon A J, Shi X P, Zhang X, Hodor P, Stone D J, Burchard J, Cavet G, Bartz S, Linsley P, Ray W J, Hazuda D. (2006) A genome wide analysis of ubiquitin ligases in APP processing identifies a novel regulator of BACE1 mRNA levels. Mol Cell Neurosci. 33(3): 227-35.

Berke, S. J., Paulson, H. L. (2003) Protein aggregation and the ubiquitin proteasome pathway: gaining the UPPer hand on neurodegeneration. Curr. Opin. Genet. Dev. 13: 253-261.

Christie, G., Markwell, R. E., Gray, C. W., Smith, L., Godfrey, F., Mansfield, F., Wadsworth, H., King, R., McLaughlin, M., Cooper, D. G.,Ward, R. V., Howlett, D. R., Hartmann, T., Lichtenthaler, S. F., Beyreuther, K., Underwood, J., Gribble, S. K., Cappai, R., Masters, C. L., Tamaoka, A., Gardner, R. L., Rivett, A. J., Karran, E. H., Allsop, D. (1999) Alzheimer's disease. Correlation of the suppression of β-Amyloid peptide secretion from cultured cells with inhibition of the chymotrypsin-like activity of the proteasome. J. Neurochem. 73: 195-204.

Flood, F., Murphy, S., Cowburn, R. F., Lannfelt, L., Walker, B., Johnston, J. A. (2005) Proteasome-mediated effects on amyloid precursor protein processing at the gamma-secretase site. Biochem. J. 385: 545-550.

Nunan, J., Shearman, M. S., Checler, F., Cappai, R., Evin, G., Beyreuther, K., Masters, C. L., Small, D. H. (2001) The C-terminal fragment of the Alzheimer's disease amyloid protein precursor is degraded by a proteasome-dependent mechanism distinct from gamma-secretase. Eur. J. Biochem. 268: 5329-5336.

Yamazaki, T., Haas, C., Takaomi, C. S., Omura, S., Ihara, Y. (1997) Specific increase in Amyloid beta protein 42 secretion ratio by calpain inhibition. Biochemistry 36: 8377-8383.

López Salon, M., Morelli, L., Castaño, E. M., Soto, E. F., Pasquini, J. M. (2000) Defective ubiquitination of cerebral proteins in Alzheimer's disease. J. Neurosci. Res. 62: 302-310.

Keller, J. N., Hanni, K. B., Markesbery, W. R. (2000) Impaired proteasome function in Alzheimer's disease. J. Neurochem. 75: 436-439.

Caporaso, G. L., S. E. Gandy, J. D. Buxbaum, and P. Greengard. (1992) Chloroquine inhibits intracellular degradation but not secretion of Alzheimer β/A4 amyloid precursor protein. Proc. Natl. Acad. Sci. USA. 89: 2252-2256.

Checler, F., C. A. da Costa, K. Ancolio, N. Chevallier, E. Lopez-Perez, and P. Marambaud. (2000) Role of the proteasome in Alzheimer's disease. Biochem. Biophys. Acta. 1502: 133-138.

Ciallella, J. R., V. V. Rangnekar, and J. P. McGillis. (1994) Heat shock alters Alzheimer's β-amyloid precursor protein expression in human endothelial cells. J. Neurosci. Res. 37: 769-776.

De Strooper, B., and W. Annaert. (2000) Proteolytic processing and cell biological functions of the amyloid precursor protein. J. Cell Sci. 113: 1857-1870.

Galbete, J. L., T. R. Martin, E. Peressini, P. Modena, R. Bianchi, and G. Forloni. (2000) Cholesterol decreases secretion of the secreted form of amyloid precursor protein by interfering with glycosylation in the protein secretory pathway. Biochem. J. 348: 307-313.

Mills, J. and P. B. Reiner. (1999) Regulation of amyloid precursor protein cleavage. J. Neurochem. 72: 443-460.

Shepherd, C. E., S. Bowes, D. Parkinson, M. Cambray-Deakin, and R. C. Pearson. (2000) Expression of amyloid precursor protein in human astrocytes in vitro: isoform-specific increases following heat shock. Neuroscience. 99: 317-325.

Schubert, D., L.-W. Jin, T. Saitoh, and G. Cole. (1989) The regulation of amyloid β protein precursor secretion and its modulatory role in cell adhesion. Neuron. 3: 689-694.

Yang, Y., R. S. Turner, and J. R. Gaut. (1998) The chaperone BiP/GRP78 binds to amyloid precursor protein and decreases Aβ40-42 secretion. J. Biol. Chem. 273: 25552-25555.

Kouchi, Z., H. Sorimachi, K. Suzukui, and S. Ishiura. (1999) Proteasome inhibitors induce the association of Alzheimer's amyloid precursor with Hsc73. Biochem. Biophys. Res. Commun. 254: 804-810.

Hare, J. F. (2001) Protease inhibitors divert amyloid precursor protein to the secretory pathway. Biochem. Biophys. Res. Commun. 281: 1298-1303.

Koo, E. H., and Squazzo, S. L. (1994) Evidence that production and release of amyloid beta-protein involves the endocytic pathway. J. Biol. Chem. 269: 17386-17389.

Hoffmann, J., Twiesselmann, C., Kummer, M. P., Romagnoli, P., and Herzog, V. (2000) A possible role for the Alzheimer amyloid precursor protein in the regulation of epidermal basal cell proliferation. Eur. J. Cell Biol., 79: 905-914.

Schmitz, A., Tikkanen, R., Kirfel, G., and Herzog, V. (2002) The biological role of the Alzheimer amyloid precursor protein in epithelial cells. Histochem. Cell Biol., 117: 171-180.

Ohsawa, I., Takamura, C., Morimoto, T., Ishiguro, M., and Kohsaka, S. (1999) Aminoterminal region of secreted form of amyloid precursor protein stimulates proliferation of neural stem cells. Eur. J. Neurosci., 11: 1907-1913.

Siemes C, Quast T, Kummer C, Wehner S, Kirfel G, Muller U, Herzog V. (2006) Keratinocytes from APP/APLP2-deficient mice are impaired in proliferation, adhesion and migration in vitro. Exp Cell Res. 312(11): 1939-49.

Lopez-Sanchez N, Muller U and Frade J M. (2005) Lengthening of G2/mitosis in cortical precursors from mice lacking beta-amyloid precursor protein. Neuroscience. 130(1): 51-60.

Caillé I, Allinquant B, Dupont E, Bouillot C, Langer A, Müller U, Prochiantz A. (2004) Soluble form of amyloid precursor protein regulates proliferation of progenitors in the adult subventricular zone. Development. 131(9): 2173-81.

Hayashi Y, Kashiwagi K, Ohta J, Nakajima M, Kawashima T, Yoshikawa K. (1994) Alzheimer amyloid protein precursor enhances proliferation of neural stem cells from fetal rat brain. Biochem Biophys Res Commun. 205(1): 936-43.

Kummer C, Wehner S, Quast T, Werner S, Herzog V. (2002) Expression and potential function of beta-amyloid precursor proteins during cutaneous wound repair. Exp Cell Res. 280(2): 222-32.

Hardy, J., Duff, K., Hardy, K. G., Perez-Tur, J., Hutton, M. (1998) Genetic dissection of Alzheimer's disease and related dementias: amyloid and its relationship to tau. Nat Neurosci 1: 355-8.

Schellenberg, G. D. (1995) Genetic dissection of Alzheimer disease, a heterogeneous disorder. Proc Natl Acad Sci USA 92: 8552-8559.

Selkoe, D. J. (1997) Alzheimer's disease: genotypes, phenotypes, and treatments. Science 275: 630-631.

Tanzi, R. E., Bush, A. I., Wasco, W. (1994) Genetic studies of Alzheimer's disease: lessons learned and future imperatives. Neurobiol Aging 15 Suppl 2: S145-S148.

Haass, C., Schlossmacher, M., Hung, A. Y., Vigo-Pelfrey, C., Mellon, A., Ostaszewski, B., Lieberburg, I., Koo, E. H., Schenk, D., Teplow, D., & Selkoe, D. J. (1992) Amyloid-β peptide is produced by cultured cells during normal metabolism. Nature 359: 322-325.

Shoji, M., Golde, T. E., Ghiso, J., Cheung, T. T., Estus, S., Shaffer, L. M., Cai, X. D., McKay, D. M., Tintner, R., Frangione, B., & Younkin, S. G. (1992) Production of the Alzheimer amyloid beta protein by normal proteolytic processing. Science 258: 126-129.

Nitsch, R. M., Slack, B. E., Wurtman, R. J., & Growdon, J. H. (1992) Release of Alzheimer amyloid precursor derivatives stimulated by activation of muscarinic acetylcholine receptors. Science 258: 304-306.

Chartier-Harlin M C, Crawford F, Hamandi K, Mullan M, Goate A, Hardy J, Backhovens H, Martin J J, Broeckhoven C V. (1991) Screening for the beta-amyloid precursor protein mutation (APP717: Val-Ile) in extended pedigrees with early onset Alzheimer's disease. Neurosci Lett. 129(1): 134-5.

Goate, A., Chartier-Harlin, M. C., Mullan, M., Brown, J., Crawford, F., et al. (1991) Segregation of a missense mutation in the amyloid precursor protein gene with familial Alzheimer's disease. Nature 349: 704-706.

Levy, E., Carman, M. D., Fernandez-Madrid, I. J., Power, M. D., Lieberburg, I., et al. (1990) Mutation of the Alzheimer's disease amyloid gene in hereditary cerebral hemorrhage, Dutch type. Science 248: 1124-1126.

Mullan, M., Crawford, F., Axelman, K., Houlden, H., Lilius, L., et al. (1992) A pathogenic mutation for probable Alzheimer's disease in the APP gene at the N-terminus of β-amyloid. Nat Genet 1: 345-347.

Murrell, J., Farlow, M., Ghetti, B., Benso (1991) A mutation in the amyloid precursor protein associated with hereditary Alzheimer's disease. Science 254: 97-99.

Nagy Z. (1999) Mechanisms of neuronal death in Down's syndrome. J. Neural Transm. Suppl. 57: 233-245.

Khurana V, Lu Y, Steinhilb M L, Oldham S, Shulman J M, Feany M B. (2006) TOR-mediated cell-cycle activation causes neurodegeneration in a Drosophila tauopathy model. Curr Biol. 16: 230-41.

Vincent, I., Zheng, J. H., Dickson, D. W., Kress, Y., Davies, P. (1998) Mitotic phosphoepitopes precede paired helical filaments in Alzheimer's disease. Neurobiol. Aging 19: 287-296.

McShea, A., Harris, P. L., Webster, K. R., Wahl, A. F., Smith, M. A. (1997) Abnormal expression of the cell cycle regulators P16 and CDK4 in Alzheimer's disease. Am. J. Pathol. 150: 1933-1939.

Busser J, Geldmacher DS, Herrup K. (1998) Ectopic cell cycle proteins predict the sites of neuronal cell death in Alzheimer's disease brain. J Neurosci.18(8): 2801-7.

Herrup K and Arendt T. (2002) Re-expression of cell cycle proteins induces neuronal cell death during Alzheimer's disease. J. Alzheimers Dis. 4: 243-247.

Yang Y, Varvel N H, Lamb B T, Herrup K. (2006) Ectopic cell cycle events link human Alzheimer's disease and amyloid precursor protein transgenic mouse models. J Neurosci. 26: 775-84.

Raina, A. K., Zhu, X., Rottkamp, C. A., Monteiro, M., Takeda, A., Smith, M. A. (2000) Cyclin toward dementia: cell cycle abnormalities and abortive oncogenesis in Alzheimer disease. J. Neurosci. Res. 61: 128-133.

Copani, A., Condorelli, F., Canonico, P. L., Nicoletti, F., Sortino, M. A. (2001) Cell cycle progression towards Alzheimer's disease. Funct. Neurol. 16: 11-15.

Arendt, T. (2002) Dysregulation of neuronal differentiation and cell cycle control in Alzheimer's disease. J. Neural Transm. Suppl. 77-85.

Bowser, R., Smith, M. A. (2002) Cell cycle proteins in Alzheimer's disease: plenty of wheels but no cycle. J. Alzheimers Dis. 4: 249-254.

Arendt T. (2003) Synaptic plasticity and cell cycle activation in neurons are alternative effector pathways: the ‘Dr. Jekyll and Mr. Hyde concept’ of Alzheimer's disease or the yin and yang of neuroplasticity. Prog Neurobiol. 71: 83-248.

Yang Y, Geldmacher D S, Herrup K. (2001) DNA replication precedes neuronal cell death in Alzheimer's disease. J Neurosci. 21: 2661-8.

Morris H R, Taylor G W, Masento M S, Jermyn K A and Kay R R. (1987) Chemical structure of the morphogen differentiation inducing factor from Dictyostelium discoideum. Nature 328: 811-814.

Ko S Y, Lin S C, Chang K W, Wong Y K, Liu C J, Chi C W, Liu T Y. (2004) Increased expression of amyloid precursor protein in oral squamous cell carcinoma. Int J Cancer. 111(5): 727-32.

Ko SY, Lin S C, Wong Y K, Liu C J, Chang K W, Liu T Y. (2007) Increase of disintergin metalloprotease 10 (ADAM10) expression in oral squamous cell carcinoma. Cancer Lett. January 8; 245(1-2): 33-43.

Meng J Y, Kataoka H, Itoh H, Koono M. (2001) Amyloid beta protein precursor is involved in the growth of human colon carcinoma cell in vitro and in vivo. Int J Cancer. 92(1): 31-9.

Wasco, W., Bupp, K., Magendantz, M., Gusella, J., Tanzi, R. E. and Solomon, F. (1992) Identification of a mouse brain cDNA that encodes a protein related to the Alzheimer disease-associated amyloid beta protein precursor. Proc. Natl. Acad. Sci. U.S.A. 89: 10758-10762.

Wasco, W., Gurubhagavatula, S., Paradis, M. D., Romano, D. M., Sisodia, S. S., Hyman, B. T., Neve, R. L. and Tanzi, R. E. (1993) Isolation and characterization of APLP2 encoding a homologue of the Alzheimer's associated amyloid beta protein precursor. Nat. Genet. 5: 95-99.

Walsh, D. M. and Selkoe, D. J. (2004) Deciphering the molecular basis of memory failure in Alzheimer's disea se. Neuron. 44(1): 181-93.

Xia W, Zhang J, Kholodenko D, Citron M, Podlisny M B, Teplow D B, Haass C, Seubert P, Koo E H, Selkoe D J. (1997) Enhanced production and oligomerization of the 42-residue amyloid beta-protein by Chinese hamster ovary cells stably expressing mutant presenilins. J Biol Chem. 272(12): 7977-82.

Nagy Z, Esiri M M, Smith A D. (1998) The cell division cycle and the pathophysiology of Alzheimer's disease. Neuroscience 87: 731-9.

Zhu, X., Rottkamp, C. A., Boux, H., Takeda, A., Perry, G., Smith, M. A. (2000) Activation of p38 kinase links tau phosphorylation, oxidative stress, and cell cycle-related events in Alzheimer disease. J. Neuropathol. Exp. Neurol. 59: 880-888.

Kessin, R. H. (2001). Dictyostelium. Cambridge: Cambridge University Press.

Thompson, C. R. L., Reichelt, S. and Kay, R. R. (2004). A demonstration of pattern formation without positional information in Dictyostelium. Dev. Growth Differ. 46: 363-369.

Kay, R. (1998) The Biosynthesis of Differentiation-Inducing Factor, a Chlorinated Signal Molecule Regulating Dictyostelium Development. 273(5): 2669-2675.

Cabrejo L, Guyant-Marechal L, Laquerriere A, Vercelletto M, De la Fourniere F, Thomas-Anterion C, Verny C, Letournel F, Pasquier F, Vital A, Checler F, Frebourg T, Campion D, Hannequin D. (2006) Phenotype associated with APP duplication in five families. Brain. 129(Pt 11): 2966-76.

Williams, J. G., Duffy, K. T., Lane, D. P., Mcrobbie, S. J., Harwood, A. J., Traynor, D., Kay, R. R. and Jermyn, K. A. (1989) Origins of the prestalk-prespore pattern in Dictyostelium development. Cell 59: 1157 -1163.

Morris, H. R., Taylor, G. W., Masento, M. S., Jermyn, K. A. and Kay, R. R. (1987) Chemical structure of the morphogen differentiation inducing factor from Dictyostelium discoideum. Nature 328: 811 -814.

Kay, R. R. and Thompson, C. R. L. (2001) Cross-induction of cell types in Dictyostelium: evidence that DIF-1 is made by prespore cells. Development 128: 4959 -4966.

Kimmel, A. and Firtel, R. A. (2004) Breaking symmetries: regulation of Dictyostelium development through chemoattractant and morphogen signal-response. Curr. Opin. Genet. Dev. 14: 540 -549

Strmecki, L., Greene, D. M. and Pears, C. (2005) Developmental decisions in Dictyostelium discoideum. Dev. Biol. 284: 25 -36.

Asahi K, Sakurai A, Takahashi N, Kubohara Y, Okamoto K and Tanaka Y. (1995) DIF-1, morphogen of Dictyostelium discoideum, induces the erythroid differentiation in murine and human leukemia cells. Biochem. Biophys. Res. Commun. 208: 1036-1039.

Kikuchi H, Oshima Y, Ichimura A, Gokan N, Hasegawa A, Hosaka K, Kubohara Y. (2006) Anti-leukemic activities of Dictyostelium secondary metabolites: a novel aromatic metabolite, 4-methyl-5-n-pentylbenzene-1,3-diol, isolated from Dictyostelium mucoroides suppresses cell growth in human leukemia K562 and HL-60 cells. Life Sci. 80(2): 160-5.

Yasmin T, Takahashi-Yanaga F, Mori J, Miwa Y, Hirata M, Watanabe Y, Morimoto S, Sasaguri T. (2005) Differentiation-inducing factor-1 suppresses gene expression of cyclin D1 in tumor cells. Biochem Biophys Res Commun. 338(2): 903-9.

Kubohara, Y. (1999) Effects of differentiation-inducing factors of Dictyostelium discoideum on human leukemia K562 cells. Eur J Pharmacol. 381(1): 57-62.

Kanai M, Konda Y, Nakajima T, Izumi Y, Kanda N, Nanakin A, Kubohara Y, Chiba T. (2003) Differentiation-inducing factor-1 (DIF-1) inhibits STAT3 activity involved in gastric cancer cell proliferation via MEK-ERK-dependent pathway. Oncogene. 22(4): 548-54.

Yang, K., Yang G., Stacey W. C., Harwalkar, J., Fretthold, J., Hitomi M., and Stacey, D. W. (2006) Glycogen synthase kinase 3 has a limited role in cell cycle regulation of cyclin D1 levels. BMC Cell Biology 7: 33.

Zhou, J., U. Liyanage, M. Medina, C. Ho, A. D. Simmons, M. Lovett, and K. S. Kosic. (1997) Presenilin 1 interaction in the brain with a novel member of the armadillo family. Neuroreport. 8: 2085-2090.

Sherrington, R., Rogaev, E.I., Liang, Y., Rogaeva, E. A., Levesque, G., Ikeda, M., Chi, H., Lin, C., Li, G., Holman, K., Tsuda, T., Mar, L., Foncin, J.-F., Bruni, A. C., Montesi, M. P., Sorbi, S., Rainero, I., Pinessi, L., Nee, L., Chumakov, I., Pollen, D., Brookes, A., Sanseau, P., Polinsky, R. J., Wasco, W., Da Silva, H. A. R., Haines, J. L., Pericak-Vance, M. A., Tanzi, R. E., Roses, A. D., Fraser, P. E., Rommens, J. M. and St. George-Hyslop, P. H. (1995) Cloning of a gene bearing missense mutations in early-onset familial Alzheimer's disease. Nature 375: 754-760.

Rogaev, E. I., Sherrington, R., Rogaeva, E. A., Levesque, G., Ikeda, M., Liang, Y., Chi, H., Lin, C., Holman, K., Tsuda, T., et al. (1995) Familial Alzheimer's disease in kindreds with missense mutations in a gene on chromosome 1 related to the Alzheimer's disease type 3 gene, Nature 376: 775-8.

Levy-Lahad, E., Wasco. W., Poorkaj, P., Romano, D. M., Oshima, J., Pettingell, W. H., Yu, C. E., Jondro, P. D., Schmidt, S. D., Wang, K., Crowley, A. C., Ying-Hui, F., Guenette, S. Y., Galas, D., Nemens, E., Wijsman, E. M., Bird, T. D., Schellenberg, G. D. and Tanzi, R. E. (1995) Candidate gene for the chromosome 1 familial Alzheimer's disease locus. Science 269: 973-977.

De Strooper, B., Saftig, P., Craessaerts, K., Vanderstichele, H., Guhde, G., Annaert, W., Von Figura, K., and Van Leuven, F. (1998) Deficiency of presenilin-1 inhibits the normal cleavage of amyloid precursor protein, Nature 391: 387-90.

Wolfe, M. S., Xia, W., Ostaszewski, B. L., Diehl, T. S., Kimberly, W. T., and Selkoe, D. J. (1999) Two transmembrane aspartates in presenilin-1 required for presenilin endoproteolysis and γ-secretase activity, Nature 398: 513-7.

Matsushime H, Quelle D, Shurtleff S A, Shibuya M, Sherr C, Kata J Y. (1994) D-type cyclin-dependent kinase activity in mammalian cells. Mol. Cell Biol. 14: 2066-2076. Diehl J A, Zindy F, Sherr C J. (1997) Inhibition of cyclin D1 phosphorylation on threonine-286 prevents its rapid degradation via the ubiquitin proteasome pathway. Genes & Development. 11: 957-972.

Morin P J. (1999) beta-catenin signaling and cancer. Bioessays. (12): 1021-30. Review.

Xia X, Qian S, Soriano S, Wu Y, Fletcher A M, Wang X J, Koo E H, Wu X, Zheng H. (2001) Loss of presenilin 1 is associated with enhanced beta-catenin signaling and skin tumorigenesis. Proc Natl Acad Sci. USA 98: 10863-10868.

Kay, R. R., and Jermyn, K. A. (1983) A possible morphogen controlling differentiation in Dictyostelium. Nature 303: 242-244.

Pastorino L, Sun A, Lu P J, Zhou X Z, Balastik M, Finn G, Wulf G, Lim J, Li S H, Li X, Xia W, Nicholson L K, Lu K P. (2006) The prolyl isomerase Pin1 regulates amyloid precursor protein processing and amyloid-beta production. Nature. 440(7083): 528-34.

Eggert S, Paliga K, Soba P, Evin G, Masters C L, Weidemann A, Beyreuther K. (2004) The proteolytic processing of the amyloid precursor protein gene family members APLP-1 and APLP-2 involves alpha-, beta-, gamma-, and epsilon-like cleavages. J Biol Chem. 279(18): 18146-56.

Walsh D M, Minogue A M, Sala Frigerio C, Fadeeva J V, Wasco W, Selkoe D J. (2007) The APP family of proteins: similarities and differences. Biochem Soc Trans. 35(Pt 2): 416-20.

Selkoe D J, Yamazaki T, Citron M, Podlisny M B, Koo E H, Teplow D B, Haass C. (1996) The role of APP processing and trafficking pathways in the formation of amyloid beta-protein. Ann N Y Acad Sci. 777: 57-64.

Lee M S, Kao S C, Lemere C A, Xia W, Tseng H C, Zhou Y, Neve R, Ahlijanian M K, Tsai L H. (2003) APP processing is regulated by cytoplasmic phosphorylation. J Cell Biol. 163(1): 83-95.

Oishi, M., A. C. Nairn, A. J. Czernik, G. S. Lim, T. Isohara, S. E. Gandy, P. Greengard, and T. Suzuki. (1997) The cytoplasmic domain of Alzheimer's amyloid precursor protein is phosphorylated at Thr654, Ser655, and Thr668 in adult rat brain and cultured cells. Mol. Med 3: 111-123.

Tarr, P. E., R. Roncarati, G. Pelicci, P. G. Pelicci, and L. D'Adamio. (2002) Tyrosine phosphorylation of the beta-amyloid precursor protein cytoplasmic tail promotes interaction with Shc. J Biol. Chem. 277: 16798-16804.

Suzuki, T., M. Oishi, D. R. Marshak, A. J. Czernik, A. C. Nairn, and P. Greengard. (1994) Cell cycle-dependent regulation of the phosphorylation and metabolism of the Alzheimer amyloid precursor protein. EMBO J. 13: 1114-1122.

Standen, C. L., J. Brownlees, A. J. Grierson, S. Kesavapany, K. F. Lau, D. M. McLoughlin, and C. C. Miller. (2001) Phosphorylation of thr(668) in the cytoplasmic domain of the Alzheimer's disease amyloid precursor protein by stress-activated protein kinase 1 b (Jun N-terminal kinase3). J. Neurochem. 76: 316-320.

Vingtdeux V, Hamdane M, Gompel M, Bégard S, Drobecq H, Ghestem A, Grosjean ME, Kostanjevecki V, Grognet P, Vanmechelen E, Buée L, Delacourte A, Sergeant N. (2005) Phosphorylation of amyloid precursor carboxy-terminal fragments enhances their processing by a gamma-secretase-dependent mechanism. Neurobiol Dis. 2005 20(2): 625-37.

Crews, C. M. and J. B. Shotwell. (2003) Small-molecule inhibitors of the cell cycle: an overview. Prog. In Cell Cycle Res. 5: 125-133.

### Equivalents

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

Those skilled in the art will recognize, or be able to ascertain using no more than routine experimentation, many equivalents of the specific embodiments of the invention described herein. Such equivalents are intended to be encompassed by the following claims.

All of the references described herein are incorporated by reference for the purposes cited herein.

1. A method for treating or preventing a disease in which Aβ is a causative factor or symptom, comprising:
administering to a subject in need of such treatment a composition comprising DIF-1, an analog thereof, a salt thereof, a solvate thereof or any combination thereof, in an amount effective to reduce Aβ production.

2. The method of claim 1, wherein the disease is Alzheimer's disease (AD), Down's syndrome, multi-infarct dementia, dementia puglistica, cerebrovascular amyloidosis (Cerebral Amyloid Angiopathy), Hereditary Amyloidosis with Cerebral Hemorrhage of the Dutch Type (HCHWA-D), Familial British Dementia, vascular dementia, and inclusion body myositis, or homozygocity for the apolipoprotein E4 allele.

3. The method of claim 2, wherein the disease is Alzheimer's disease.

4. The method of claim 1, wherein the subject is a human.

5. The method of claim 1, wherein the subject is otherwise free of symptoms calling for treatment with the agent.

6. The method of claim 1, wherein the subject does not have a cancer.

7. The method of claim 1, wherein the subject is apparently healthy.

8. The method of claim 1, wherein the subject exhibits one or more symptoms of a disease in which Aβ is a causative factor or symptom.

9. The method of claim 8, wherein the disease in which Aβ is a causative factor or symptom is Alzheimer's disease (AD), Down's syndrome, multi-infarct dementia, dementia puglistica, cerebrovascular amyloidosis (Cerebral Amyloid Angiopathy), Hereditary Amyloidosis with Cerebral Hemorrhage of the Dutch Type (HCHWA-D), Familial British Dementia, vascular dementia, and inclusion body myositis, or homozygocity for the apolipoprotein E4 allele.

10. The method of claim 1, wherein Aβ production is reduced by at least 10%.

11. The method of claim 1, wherein Aβ production is reduced by at least 20%.

12. The method of claim 1, wherein Aβ production is reduced by at least 50%.

13. The method of claim 3, further comprising administering an Alzheimer's disease treatment.

14. The method of claim 13, wherein the Alzheimer's disease treatment is a cholinesterase inhibitor; a NMDA receptor antagonist; an AMPA receptor agonist; a choline uptake enhancer; a HMG CoA reductase inhibitor; or immune therapy.

15. The method of claim 14, wherein the cholinesterase inhibitor is donepezil (Aricept®), rivastigmine (Exelon®), galantamine (Reminyl®), or tacrine (Cognex®).

16. The method of claim 14, wherein the NMDA receptor antagonist is memantine (Namenda®).

17. The method of claim 14, wherein the AMPA receptor agonist is CX516 (Ampalex®).

18. The method of claim 14, wherein the choline uptake enhancer is MKC-231.

19. The method of claim 14, wherein the HMG CoA reductase inhibitor is a statin.

20. The method of claim 3, wherein the DIF-1, analog thereof, salt thereof, solvate thereof, Alzheimer's disease therapeutic, or combination thereof is administered orally, intravenously, intramuscularly, intranasally, intraperitoneally, subcutaneously, or intrathecally.

21-53. (canceled)
